(19) United States
(12) Patent Application Publication
(10) Pub. No.: US 2024/0426827 A1
(43) Pub. Date: Dec. 26, 2024

(54) ANTI-HUMAN CD10 ANTIBODIES FOR USE IN IMMUNOHISTOCHEMISTRY (IHC) PROTOCOLS TO DIAGNOSE CANCER

(71) Applicant: Agilent Technologies, Inc., Santa Clara, CA (US)

(72) Inventors: Morten Draeby SORENSEN, Herlev (DK); Tine HAGEDORN-OLSEN, Skibby (DK)

(21) Appl. No.: 18/028,190

(22) PCT Filed: Dec. 1, 2021
(86) PCT No.: PCT/US2021/061422

§ 371 Date: Mar. 23, 2023

Related U.S. Application Data
(60) Provisional application No. 63/120,404, filed on Dec. 2, 2020.

(51) Int. Cl. G01N 33/574; C07K 16/28

(52) U.S. Cl. CPC G01N 33/57492; C07K 16/2896; C07K 2317/31; C07K 2317/569; C07K 2317/622; C07K 2317/626; C07K 2319/60; G01N 2333/70596; G01N 2474/20

(57) ABSTRACT

In alternative embodiments, provided are non-natural synthetic antibodies capable of specifically binding a human CD10 polypeptide, or neprilysin, polypeptide. In alternative embodiments, also provided are products of manufacture and kits comprising antibodies as provided herein, and methods for making and using antibodies as provided herein, where the antibodies can be used for in vitro diagnostics by immunohistochemistry (IHC). In alternative embodiments, antibodies as provided herein are used in IHC protocols to diagnose a cancer, for example, leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, myeloma, precursor B lymphoblastic leukemia or lymphoma, follicular lymphoma, mantle cell lymphoma, precursor T lymphoblastic leukemia or lymphoma, non-hematolymphoid sarcoma, or carcinoma such as renal cell carcinoma or metaplastic breast carcinoma.

### RELATED APPLICATIONS

This Patent Convention Treaty (PCT) International Patent Application claims the benefit of priority under 35 U.S.C. § 119(e) to U.S. Provisional Patent Application Serial No. (USSN) 63/120,404, filed Dec. 2, 2020. The aforementioned applications are expressly incorporated herein by reference in their entirety and for all purposes.

### TECHNICAL FIELD

This invention generally relates to immunohistochemistry (IHC) and cancer diagnosis. In alternative embodiments, provided are non-natural, or synthetic, antibodies capable of specifically binding a human CD10, or neprilysin, polypeptide. In alternative embodiments, also provided are products of manufacture and kits comprising antibodies as provided herein, and methods for making and using antibodies as provided herein, where the antibodies can be used for in vitro diagnostics by immunohistochemistry (IHC). In alternative embodiments, antibodies as provided herein are used in IHC protocols to diagnose a cancer, for example, leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, myeloma, precursor B lymphoblastic leukemia or lymphoma, follicular lymphoma, diffuse large B-cell lymphoma, mantle cell lymphoma, precursor T lymphoblastic leukemia or lymphoma, non-hematolymphoid sarcoma, or carcinoma such as renal cell carcinoma or metaplastic breast carcinoma.

### BACKGROUND

Neprilysin, also known as cluster of differentiation 10 (CD10), membrane metallo-endopeptidase (MME), neutral endopeptidase (NEP), and common acute lymphoblastic leukemia antigen (CALLA), is an enzyme that in humans is encoded by the MME gene. Neprilysin is a zinc-dependent metalloprotease that cleaves peptides at the amino side of hydrophobic residues and inactivates several peptide hormones including glucagon, enkephalins, substance P, neurotensin, oxytocin, and bradykinin. It also degrades the amyloid beta peptide whose abnormal folding and aggregation in neural tissue has been implicated as a cause of Alzheimer's disease. Synthesized as a membrane-bound protein, the neprilysin ectodomain is released into the extracellular domain after it has been transported from the Golgi apparatus to the cell surface.

CD10 is of use in hematological diagnosis since it is expressed by early B, pro-B and pre-B lymphocytes, and by lymph node germinal center cells. Hematopoietic and lymphoid tissue diseases in which it is positive include ALL, angioimmunoblastic T cell lymphoma, Burkitt lymphoma, chronic myelogenous leukemia in blast crisis (90%), diffuse large B-cell lymphoma (variable), follicular lymphoma (70%), hairy cell leukemia (10%), and myeloma (some). CD10 tends to be negative in acute myeloid leukemia, chronic lymphocytic leukemia, mantle cell lymphoma, and marginal zone lymphoma. CD10 is found on non-T ALL cells, which derive from pre-B lymphocytes, and in germinal center-related non-Hodgkin lymphoma such as Burkitt lymphoma and follicular lymphoma, but not on leukemia cells or lymphomas, which originate in more mature B cells.

### SUMMARY

In alternative embodiments, provided are non-natural, or synthetic, antibodies capable of specifically binding a human CD10 or neprilysin polypeptide. In alternative embodiments, which can be used in immunohistochemistry (IHC) assays.

In alternative embodiments, provided are isolated or purified antibodies (Ab), or antigen (Ag) binding fragments thereof, or monomeric or dimeric antigen binding proteins (ABP), capable of specifically binding a human CD10 polypeptide (also called neprilysin, membrane metallo-endopeptidase (MME), neutral endopeptidase (NEP) and common acute lymphoblastic leukemia antigen (CALLA)) or CD10 peptide), wherein the isolated or purified Ab, or Ag binding fragment thereof, or monomeric or dimeric ABP comprises:
(a) an immunoglobulin heavy chain variable region comprising:
   (i) an amino acid sequence comprising the three CDR1, CDR2 and CDR3 complementarity determining regions (CDRs) of SEQ ID NO:1, or CDR1 amino acid (aa) residues GYTFTDYF (residues 26-33 of SEQ ID NO:1), CDR2 aa residues INPNNGDT (residues 51 to 58 of SEQ ID NO:1), and CDR3 aa residues AKGGFNPGDY (residues 97-106 of SEQ ID NO:1), or
   (ii) amino acid sequences having at least about 70%, 75%, 80%, 85%, 90%, 95%, 98% sequence identity to each of the three CDR1, CDR2 and CDR3 complementarity determining regions (CDRs) of SEQ ID NO:1, or CDR1 amino acid (aa) residues GYTFTDYF (residues 26-33 of SEQ ID NO:1), CDR2 aa residues INPNNGDT (residues 51 to 58 of SEQ ID NO:1), and CDR3 aa residues AKGGFNPGDY (residues 97-106 of SEQ ID NO:1), or
   (iii) an amino acid sequence having at least about 70%, 75%, 80%, 85%, 90%, 95%, 98% sequence identity to SEQ ID NO:1, or an amino acid sequence having complete sequence identity to SEQ ID NO:1;
(b) an immunoglobulin light chain variable region comprising:
   (i) an amino acid sequence comprising the three CDR1, CDR2 and CDR3 complementarity determining regions (CDRs) of SEQ ID NO:2, or CDR1 amino acid (aa) residues QSLVHRNGNTY (residues 27-37 of SEQ ID NO:2), CDR2 aa residues KVS (residues 55 to 57 of SEQ ID NO:2), and CDR3 aa residues SQSTHVPLT (residues 94-102 of SEQ ID NO:2), or
   (ii) amino acid sequences having at least about 70%, 75%, 80%, 85%, 90%, 95%, 98% sequence identity to each of the three CDR1, CDR2 and CDR3 complementarity determining regions (CDRs) of SEQ ID NO:2, or CDR1 amino acid (aa) residues QSLVHRNGNTY (residues 27-37 of SEQ ID NO:2), CDR2 aa residues KVS (residues 55 to 57 of SEQ ID NO:2), and CDR3 aa residues SQSTHVPLT (residues 94-102 of SEQ ID NO:2); or
   (iii) an amino acid sequence having at least about 70%, 75%, 80%, 85%, 90%, 95%, 98% sequence identity to SEQ ID NO:2, or an amino acid sequence having complete (100%) sequence identity to SEQ ID NO:2; or
(c) the immunoglobulin heavy chain variable region of (a) and the immunoglobulin light chain variable region of (b).

In alternative embodiments, the isolated or purified Ab, or Ag binding fragment thereof, or monomeric or dimeric ABP, as provided herein are fabricated as or in the form of:
an antigen-binding fragment (Fab, or an Ab fragment having just one constant and one variable domain of each of an Ab heavy and light chain),
a F(ab′)₂ (or an Ab digested by pepsin yielding two fragments: a F(ab′)₂ fragment and a pFc′ (pepsin cleavage Fc) fragment),
a Fab′ (a single chain of a F(ab′)₂ fragment),
a single-chain variable fragment (scFv) (or a fusion protein of a variable region of an Ab heavy and light chain connected together with a linker peptide optionally of about ten to about 25 amino acids in length),
a (scFv)₂, or a di-scFv or a bi-scFv, or a single peptide chain having two variable heavy and two variable light regions yielding tandem scFv,
a minibody (or a fusion protein of a variable region of an Ab heavy and light chain connected together with an alkyl group, optionally a methyl or an ethyl group)
a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
a single-domain antibody (dAB) (or a single variable region of an Ab heavy or Ab light chain),
a plurality of complementarity determining region (CDR) fragments, or
a multispecific antibody formed from two or more antibody fragments.

In alternative embodiments of the isolated or purified Ab, or Ag binding fragment thereof, or monomeric or dimeric ABP, as provided herein, the immunoglobulin heavy chain variable region comprises:
an amino acid sequence:

### EVQLQQSGPDLVKPGASVKMSCKASGYTFTDYFMKWVKQSHGKSLEWIGDINP NNGDTFYNQKFKGKATLTVDKSSSTAYMQLSSLTSEDSAVYYCAKGGFNPGDY WGQGTTLTVSS (SEQ ID NO:1), or

SEQ ID NO:1 having one or more (for example: two, three, four, five, six, seven, eight, nine or ten or more) amino acid substitutions, additions (insertions) or deletions, and the Ab, or Ag binding fragment thereof, or monomeric or dimeric ABP (including synthetic or recombinant forms thereof) retains its ability to specifically bind to a human CD10 protein or polypeptide, and optionally the one or more (for example: two, three, four, five, six, seven, eight, nine or ten or more) amino acid substitutions comprise one or more conservative amino acid substitutions.

In alternative embodiments of the isolated or purified Ab, or Ag binding fragment thereof, or monomeric or dimeric ABP, as provided herein:
the immunoglobulin light chain variable region comprises an amino acid sequence:

### DAVLTQAPLSLPVSLGDQASISCRSSQSLVHRNGNTYLHWFLQRPGQSPKLLIDK VSNRFSGVPDRFSGSGSGTDFTLKISRVEAEDLGVYFCSQSTHVPLTFGAGTKLEL K (SEQ ID NO:2), or

SEQ ID NO:2 having one or more (for example: two, three, four, five, six, seven, eight, nine or ten or more) amino acid substitutions, additions (insertions) or deletions, and the Ab, or Ag binding fragment thereof, or monomeric or dimeric ABP (including synthetic or recombinant forms thereof) retains its ability to specifically bind to a human CD10 protein or polypeptide, and optionally the one or more (for example: two, three, four, five, six, seven, eight, nine or ten or more) amino acid substitutions comprise one or more conservative amino acid substitutions.

In alternative embodiments of the isolated or purified Ab, or Ag binding fragment thereof, or monomeric or dimeric ABP, as provided herein: SEQ ID NO:1 or SEQ ID NO:2 has two, three, four, five, six, seven, eight, nine, ten, eleven, twelve thirteen, fourteen or fifteen conservative amino acid substitutions, and the Ab, or Ag binding fragment thereof, or monomeric or dimeric ABP (including synthetic or recombinant forms thereof) retains its ability to specifically bind to a human CD10 protein or polypeptide.

In alternative embodiments of the isolated or purified Ab, or Ag binding fragment thereof, or monomeric or dimeric ABP, as provided herein:
the immunoglobulin light chain variable region further comprises at least a portion of a light chain constant region, wherein optionally the immunoglobulin light chain variable region further comprises at least about 1%, 5%, 10%, 15%, 20%, 25%, 30%, 35%, 40%, 45%, 50%, 55%, 60%, 65%, 70%, 75%, 80%, 85%, 90% or 95% or more of a light chain constant region;
the immunoglobulin heavy chain variable region further comprises at least a portion of an immunoglobulin heavy chain constant region, wherein optionally the immunoglobulin heavy chain variable region further comprises at least about 1%, 5%, 10%, 15%, 20%, 25%, 30%, 35%, 40%, 45%, 50%, 55%, 60%, 65%, 70%, 75%, 80%, 85%, 90% or 95% or more of an immunoglobulin heavy chain constant region;
the immunoglobulin light chain variable region further comprises at least a portion of an immunoglobulin light chain constant region, and the immunoglobulin heavy chain variable region further comprises at least a portion of an immunoglobulin heavy chain constant region;
the immunoglobulin heavy chain constant region comprises an amino acid sequence from a IgG, IgM, IgA, IgD or IgE isotype;
the light chain constant region comprises amino acid sequence from a kappa (κ) or lambda (λ) isotype;
the at least a portion of (or at least about 1%, 5%, 10%, 15%, 20%, 25%, 30%, 35%, 40%, 45%, 50%, 55%, 60%, 65%, 70%, 75%, 80%, 85%, 90% or 95% or more of) the heavy chain constant region, at least a portion of (or at least about 1%, 5%, 10%, 15%, 20%, 25%, 30%, 35%, 40%, 45%, 50%, 55%, 60%, 65%, 70%, 75%, 80%, 85%, 90% or 95% or more of) the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat;
at least a portion of (or at least about 1%, 5%, 10%, 15%, 20%, 25%, 30%, 35%, 40%, 45%, 50%, 55%, 60%, 65%, 70%, 75%, 80%, 85%, 90% or 95% or more of) the heavy chain constant region, at least a portion of (or at least about 1%, 5%, 10%, 15%, 20%, 25%, 30%, 35%, 40%, 45%, 50%, 55%, 60%, 65%, 70%, 75%, 80%, 85%, 90% or 95% or more of) the light chain constant region, or at least a portion of the heavy chain constant region and the light chain constant region, is or comprises a synthetic amino acid sequence;
the Ab, the Ag binding fragment thereof, or monomeric or dimeric ABP, or the heavy chain constant region, or the light chain constant region, or the heavy chain constant region and the light chain constant region, including synthetic or recombinant forms thereof, further comprise or comprises or is bound to a heterologous protein, peptide, or a compound or a composition,
and optionally the compound or a composition, comprises a detectable protein, a detectable agent or a binding moiety; and optionally the heterologous protein or peptide comprises a carrier protein; and optionally the heterologous protein, peptide or the compound or composition, is covalently conjugated to the antibody (Ab), or Ag binding fragment thereof, or monomeric or dimeric ABP (or synthetic or recombinant forms thereof); and optionally the detectable agent or binding moiety comprises a biotin, a fluorescent or chemiluminescent label, a fluorophore, perylene, fluorenyl, coumarin, 7-methoxycoumarin (Mca), 4-(dimethylaminoazo)benzene-4-carboxylic acid (dabcyl), Tamra, boron-dipyrromethene (BODIPY), or derivatives thereof, a dye, a radioisotope, a quantum dot or photoluminescent aqueous nanocrystal, a hapten, or an antibody binding epitope or domain; and optionally the dye is or comprises rhodamine, [2-(4-nitro-2,1,3-benzoxadiazol-7-yl)aminoethyl]trimethylammonium (NBD), nile red or nile blue, or is a fluorescent dye comprising sulfoindocyanine; and optionally the fluorophore is or comprises a dansyl, a fluorescein, a carboxyfluorescein (FAM) or a 6-FAM moiety; and optionally the dye is or comprises a cyanine dye, a Cy3 or a Cy5; and optionally the hapten is or comprises a biotin, a theophylline, a digoxigenin, a carborane, a fluorescein or a bromodeoxyuridine moiety; and/or
the Ab, or Ag binding fragment thereof, or monomeric or dimeric ABP is a recombinant Ab, or Ag binding fragment thereof, or monomeric or dimeric ABP, or comprises a peptide or polypeptide made by a recombinant technique.

In alternative embodiments, provided are chimeric or recombinant nucleic acids comprising: a nucleic acid sequence encoding a Ab, or Ag binding fragment thereof, or monomeric or dimeric ABP as provided herein.

In alternative embodiments, of chimeric or recombinant nucleic acids as provided herein:
the chimeric or recombinant nucleic acid further comprises and is operatively linked to a transcriptional regulatory element; and/or
the transcriptional regulatory element comprises a promoter, and optionally the promoter is an inducible promoter or a constitutive promoter.

In alternative embodiments, provided are expression cassettes, vectors, recombinant viruses, artificial chromosomes, cosmids or plasmids comprising a chimeric or a recombinant nucleic acid as provided herein.

In alternative embodiments, provided are cells (for example, recombinantly engineered cells) comprising a chimeric or recombinant antibody or dimeric antigen binding protein as provided herein, a chimeric or recombinant nucleic acid as provided herein, or an expression cassette, vector, recombinant virus, artificial chromosome, cosmid or plasmid as provided herein; and optionally the cell is a bacterial, fungal, mammalian, yeast, insect or plant cell, or optionally the mammalian cell is a human cell. In alternative embodiments, the cells are hybridoma cells capable of synthesizing (making) a monoclonal chimeric or recombinant antibody or dimeric antigen binding protein as provided herein, and/or a chimeric or recombinant nucleic acid as provided herein. In alternative embodiments, the cells are hybridoma cells capable of secreting monoclonal chimeric or recombinant antibodies or dimeric antigen binding proteins as provided herein.

In alternative embodiments, provided are methods for detecting the presence of a human CD10 protein in or on a cell, a tissue, an organ or a portion of any of the foregoing, comprising: (a) contacting the cell, tissue or organ or portion of any of the foregoing with an Ab, or Ag binding fragment thereof, or monomeric or dimeric ABP as provided herein, and, (b) detecting the specific binding of the Ab, or Ag binding fragment thereof, or monomeric or dimeric ABP with a CD10 polypeptide, or a polypeptide or peptide comprising an antigen or an epitope consisting of an amino acid sequence as set forth in SEQ ID NO:1, in or on the cell, tissue or organ or portion of any of the foregoing, thereby detecting the presence of the human CD10 protein in or on the cell, tissue, organ or portion of any of the foregoing.

In alternative embodiments of methods for detecting the presence of a human CD10 protein in or on a cell, a tissue, an organ or a portion of any of the foregoing as provided herein:
the contacting comprises use of an immunohistochemistry (IHC) assay;
the method further comprises contacting the Ab, or Ag binding fragment thereof, or monomeric or dimeric ABP, specifically bound a CD10 polypeptide, or a polypeptide or peptide comprising an antigen or an epitope consisting of an amino acid sequence as set forth in SEQ ID NO:1, with a detectable agent to indicate or signal the specific binding of the Ab, or Ag binding fragment thereof, or monomeric or dimeric ABP, to the human CD10 protein;
the detectable agent specifically binds to the Ab, or Ag binding fragment thereof, or monomeric or dimeric ABP;
the cell, tissue, organ or a portion of any of the foregoing is or comprises: an early B cell, a pro-B cell, a pre-B lymphocyte, a mature B-lymphocyte, a follicular center cell, or a cell in a lymph node germinal center, a bone marrow stem cell, a myelopoietic cell, a parafollicular T lymphocyte, a subpopulation of parafollicular T lymphocytes, a liver bile canalicular cell, a renal glomerular cell, a proximal tubular cell, a breast myoepithelial cell, a stromal cell around or associated with an infiltrating tumor cell, a kidney cell, an epithelial cell, a leukemia cell or a carcinoma cell;
the epithelial cell is a lung, intestine, kidney, breast or placental epithelial cell;
the leukemia cell is an acute lymphoblastic leukemia (ALL) cell; and/or
the carcinoma cell is a tumor cell derived from various epithelial cells, wherein optionally the carcinoma cell is a basal cell carcinoma (BCC) cell.

In alternative embodiments, provided are methods for detecting or diagnosing a cancer, wherein the method comprises detecting expression or presence of a human CD10 protein or peptide in or on a cell, tissue or organ sample using a method as provided herein, wherein the detecting of the specific binding of the Ab, or Ag binding fragment thereof, or monomeric or dimeric ABP with a CD10 polypeptide, or a polypeptide or peptide comprising an antigen or an epitope consisting of an amino acid sequence as set forth in SEQ ID NO:1, in or on the cell, tissue or organ or portion of any of the foregoing, detects or diagnoses, or assists in the detection or diagnosis of, the cancer.

In alternative embodiments of methods for detecting or diagnosing a cancer as provided herein:
the cancer is: a leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, myeloma, precursor B lymphoblastic leukemia or lymphoma, follicular lymphoma, Burkitt's lymphoma, mantle cell lymphoma, precursor T lymphoblastic leukemia or lymphoma, non-hematolymphoid sarcoma, or carcinomasuch as a renal cell carcinoma or metaplastic breast carcinoma;
the carcinoma cell is a tumor cell derived from various epithelial cells, wherein optionally the carcinoma cell is a basal cell carcinoma (BCC) cell;
the cell, tissue or organ sample is from an individual in need thereof; and/or
the detection comprises conducting an immunohistochemistry (IHC) assay.

In alternative embodiments, provided are methods for treating, ameliorating or preventing a cancer comprising first detecting or diagnosing the cancer using a method as provided herein, followed by treatment of the individual in need thereof for the treatment, amelioration or prevention of the cancer.

In alternative embodiments of methods for treating, ameliorating or preventing a cancer as provided herein: the cancer is: leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, a myeloma, a precursor B lymphoblastic leukemia or lymphoma, a follicular lymphoma, mantle cell lymphoma, precursor T lymphoblastic leukemia or lymphoma, non-hematolymphoid sarcoma, or carcinomasuch as a renal cell carcinoma or a metaplastic breast carcinoma; and optionally the carcinoma cell is a tumor cell derived from various epithelial cells, wherein optionally the carcinoma cell is a basal cell carcinoma (BCC) cell.

In alternative embodiments, provided are uses of an antibody (Ab), or antigen (Ag) binding fragment thereof, or monomeric or dimeric antigen binding protein (ABP) as provided herein (or synthetic or recombinant forms thereof) for detecting or diagnosing a cancer, or treating, ameliorating or preventing a cancer.

In alternative embodiments of uses as provided herein:
the cancer is: leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, myeloma, precursor B lymphoblastic leukemia or lymphoma, follicular lymphoma, mantle cell lymphoma, precursor T lymphoblastic leukemia or lymphoma, non-hematolymphoid sarcoma, or carcinomasuch as a renal cell carcinoma or a metaplastic breast carcinoma, and optionally the carcinoma cell is a tumor cell derived from various epithelial cells, wherein optionally the carcinoma cell is a basal cell carcinoma (BCC) cell; and/or
the detection comprises conducting an immunohistochemistry (IHC) assay,

In alternative embodiments, provided are antibodies (Ab), or antigen (Ag) binding fragments thereof, or monomeric or dimeric antigen binding proteins (ABP) as provided herein (or synthetic or recombinant forms thereof) for use in detecting or diagnosing a cancer, or treating, ameliorating or preventing a cancer; and optionally the cancer is: leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, myeloma, precursor B lymphoblastic leukemia or lymphoma, follicular lymphoma, mantle cell lymphoma, precursor T lymphoblastic leukemia or lymphoma, a non-hematolymphoid sarcoma, or carcinomasuch as a renal cell carcinoma or a metaplastic breast carcinoma, and optionally the carcinoma cell is a tumor cell derived from various epithelial cells, wherein optionally the carcinoma cell is a basal cell carcinoma (BCC) cell; and optionally the detecting or diagnosing comprises conducting an immunohistochemistry (IHC) assay.

A kit comprising: an isolated or purified antibody (Ab), or antigen (Ag) binding fragment thereof, or monomeric or dimeric antigen binding protein (ABP), capable of specifically binding a human CD10, as provided herein (or synthetic or recombinant forms thereof); a chimeric or a recombinant nucleic acid as provided herein; or an expression cassette, vector, recombinant virus, artificial chromosome, cosmid or plasmid as provided herein; or, a cell as provided herein; and optionally the kit comprises or further comprises components needed for an immunohistochemistry (IHC) assay, and/or comprises instructions for practicing a method as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1-15: illustrate immunohistochemistry (IHC) imaging capabilities of the mouse monoclonal antibody 56C6 (ABCAM, Burlingame, CA) versus the exemplary antibody Mab3124:

FIG. 1 illustrates an IHC 100× image of tonsil tissue sample;

FIG. 2 illustrates an IHC 100× image of kidney tissue sample;

FIG. 3 illustrates an IHC 100× image of liver tissue sample;

FIG. 4 illustrates an IHC 100× image of pancreas tissue sample;

FIG. 5 illustrates an IHC 100× image of a Burkitt's lymphoma tissue section;

FIG. 6 illustrates an IHC 100× image of a Burkitt's lymphoma tissue section;

FIG. 7 illustrates an IHC 100× image of a diffuse large B-cell lymphoma (DLBCL) tissue sample;

FIG. 8 illustrates an IHC 100× image of a DLBCL tissue sample;

FIG. 9 illustrates an IHC 100× image of a follicular lymphoma sample;

FIG. 10 illustrates an IHC 100× image of a plasmacytoma sample;

FIG. 11 illustrates an IHC 100× image of a renal clear cell carcinoma sample;

FIG. 12 illustrates an IHC 100× image of a renal clear cell carcinoma sample;

FIG. 13 illustrates an IHC 100× image of an ovarian endometrioid carcinoma sample;

FIG. 14 illustrates an IHC 100× image of a Hodgkin's mixed cellularity tissue sample; and

FIG. 15 illustrates an IHC 100× image of a colon adenocarcinoma issue sample, as discussed in further detail in Example 1, below.

FIG. 16-20 illustrate images showing IHC staining of CD10 tissue slides with CD10 Mab 3124, optimal protocol on AUTOSTAINER LINK™:

FIG. 16 illustrates an image of tonsil tissue stained with CD10 Mab 3124, using optimal protocol on AUTOSTAINER LINK™;

FIG. 17 illustrates an image of kidney tissue stained with CD10 Mab 3124, using optimal protocol on AUTOSTAINER LINK™;

FIG. 18 illustrates an image of a renal clear cell carcinoma stained with CD10 Mab 3124, using optimal protocol on AUTOSTAINER LINK™;

FIG. 19 illustrates an image of Burkitt lymphoma stained with CD10 Mab 3124, using optimal protocol on AUTOSTAINER LINK™; and

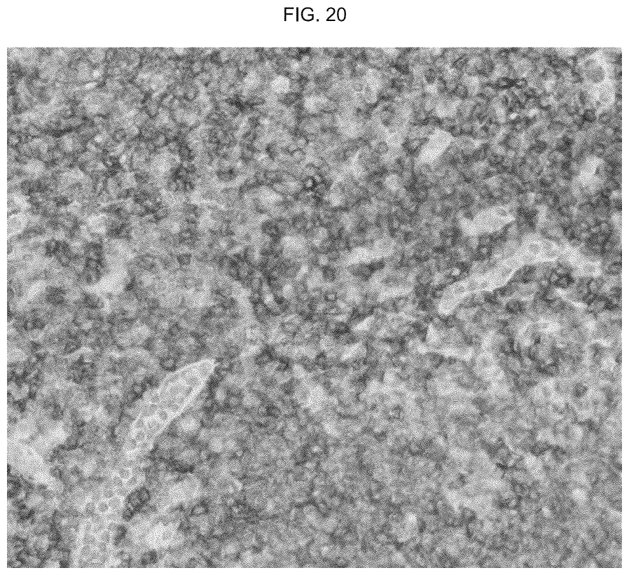

FIG. 20 illustrates an image of tonsil tissue stained with CD10 Mab 3124, using optimal protocol on AUTOSTAINER LINK™
as discussed in further detail in Example 2, below.

Like reference symbols in the various drawings indicate like elements.

### DETAILED DESCRIPTION

In alternative embodiments, provided are non-natural, or synthetic, antibodies capable of specifically binding a human CD10, or neprilysin, polypeptide. In alternative embodiments, also provided are products of manufacture and kits comprising antibodies as provided herein, and methods for making and using antibodies as provided herein. In alternative embodiments, the antibodies are used for in vitro diagnostics by immunohistochemistry (IHC), which can be used in the diagnosis and treatment of cancer and other diseases and conditions.

The anti-CD10 antibodies as provided herein were developed using a new approach for antigen design. Previous attempts were unable to develop CD10 specific antibodies functional in IHC. The newly designed antigen has yielded a very good immune response in both mice and rabbits. Three clones were developed from mice using traditional hybridoma technology and tested further for robustness, specificity and usability for in vitro diagnostic devices (IVD) by IHC. One hybridoma clone from the fusions F461/7D5/H8/B7/G8 was found to be the best of the three and is described herein.

### Expression of Recombinant Antibodies and ABPs

In alternative embodiments, provided are isolated or purified antibodies (Ab), or antigen (Ag) binding fragments thereof, or monomeric or dimeric antigen binding proteins (ABP), capable of specifically binding a human CD10 polypeptide (also called neprilysin, which can be recombinant antibodies (Abs) or antigen binding fragments thereof, or monomeric or dimeric antigen binding proteins as provided herein, including the exemplary recombinant anti-human CD10 (neprilysin) Abs comprising immunoglobulin heavy chain variable region SEQ ID NO:1 and immunoglobulin light chain variable region SEQ ID NO:2.

In alternative embodiments, recombinant antibodies (for example, genetically engineered Abs) as provide herein can comprise a signal peptide or other heterologous peptide or tag; and can be expressed as a recombinant polypeptide or Ab using any expression vehicle or expression system, for example, a vector such as a viral vector, a phage, a plasmid or a cosmid.

In alternative embodiments, a constant heavy chain is expressed together with a light chain, for example, a heavy chain can be expressed with a kappa-1 light chain.

In alternative embodiments, nucleic acids encoding the respective heavy and light chains, or the heavy chain and the light chain, are encoded in separate expression vehicles, or in the same expression vehicle or expression system.

In some embodiments, the recombinant antibodies (Abs) or antigen binding fragments thereof, or monomeric or dimeric antigen binding proteins as provided herein, including the heavy and light chains can be (cis- or trans-) as provided herein, are expressed from a pTT5™ vector(s) (National Research Council Canada, NRC-CNRC, Canada) or equivalents.

In alternative embodiments, the expression vehicles (such as a vector, plasmid virus or phage) containing nucleic acids encoding recombinant antibodies (Abs) or antigen binding fragments thereof, or monomeric or dimeric antigen binding proteins as provided herein are expressed in in vitro expression systems or are expressed in cultured tissues or cells, for example, they are expressed in a human embryonic kidney (HEK) cell such as an HEK293-6E cell.

In alternative embodiment, the expression vehicle(s), for example, a vector or vectors, expressing the recombinant antibodies (Abs) or antigen binding fragments thereof, or monomeric or dimeric antigen binding proteins as provided herein, including heavy and/or light chains, are episomal or are chromosomally integrated, for example, in a stable cell line capable of synthesizing, optionally inducible synthesizing, the recombinant antibodies (Abs) or antigen binding fragments thereof, or monomeric or dimeric antigen binding proteins as provided herein, or heavy and/or light chains as provided herein.

In alternative embodiments, provided are nucleic acids encoding recombinant antibodies (Abs) or antigen binding fragments thereof, or monomeric or dimeric antigen binding proteins as provided herein. Nucleic acids as provided herein can be made, isolated and/or manipulated by, for example, cloning and expression of cDNA libraries, amplification of message or genomic DNA by PCR, and the like. Nucleic acids used to practice embodiments as provided herein, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, fungal, mammalian, yeast, insect or plant cell expression systems, or hybrid or synthetic expression systems.

Alternatively, these nucleic acids can be synthesized in vitro by well-known chemical synthesis techniques, as described in, for example, Martin et al, ACS Synth. Biol. (2017) 6, 7, 1370-1379; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, for example, subcloning, labeling probes (for example, random-primer labeling using Klenow polymerase, nick translation, amplification), sequencing, hybridization and the like are well described in the scientific and patent literature, see, for example, Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice embodiments as provided herein comprises screening and re-cloning inserts isolated or amplified from, for example, genomic clones or cDNA clones. Sources of nucleic acids include recombinant nucleic acid sequences, genomic or cDNA libraries contained and/or expressed in, for example, mammalian artificial chromosomes (MACs), see, for example, U.S. Pat. Nos. 5,721,118; 6,025,155; human artificial chromosomes, see, for example, Rosenfeld (1997) Nat. Genet. 15:333-335; yeast artificial chromosomes (YAC); bacterial artificial chromosomes (BAC); P1 artificial chromosomes, see, for example, Woon (1998) Genomics 50:306-316; P1-derived vectors (PACs), see, for example, Kern (1997) Biotechniques 23:120-124; cosmids, recombinant viruses, phages or plasmids.

In alternative embodiments, nucleic acids as provided herein are operably linked to transcriptional regulatory elements, including promoters, with can be constitutive or inducible transcriptional regulatory elements.

In alternative aspects, provided are “expression cassettes” comprising a nucleotide sequence as provided herein, for example encoding a recombinant antibody as provided herein. Expression cassettes can include at least a transcriptional regulatory element, for example, a promoter, operably linked with an antibody coding sequence, and optionally can also include transcription termination signals. Additional factors necessary or helpful in effecting expression may also be used, for example, enhancers.

In alternative aspects, expression cassettes used to practice embodiments as provided herein include plasmids, expression vectors, recombinant viruses, any form of recombinant “naked DNA” vector, and the like. In alternative aspects, an expression vehicle or a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (for example, a cell membrane, a viral lipid envelope, etc.). In alternative aspects, vectors used to practice embodiments as provided herein can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host's genome.

In alternative aspects, “promoters” used to practice embodiments as provided herein include all sequences capable of driving transcription of a coding sequence in a cell, for example, a bacterial, yeast, fungal, plant, insect (for example, baculovirus) or mammalian cell. Thus, promoters used in the constructs include cis-acting transcriptional control elements and regulatory sequences that are involved in regulating or modulating the timing and/or rate of transcription of a gene. For example, a promoter used to practice embodiments as provided herein can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription.

“Constitutive” promoters used to practice embodiments as provided herein can be those that drive expression continuously under most environmental conditions and states of development or cell differentiation. “Inducible” or “regulatable” promoters used to practice embodiments as provided herein can direct expression of a nucleic acid as provided herein under the influence of environmental conditions or developmental conditions. Examples of environmental conditions that may affect transcription by inducible promoters used to practice embodiments as provided herein include the presence of an inducing factor administered to a cell.

In alternative embodiments, polypeptides, including recombinant antibodies (Abs) or antigen binding fragments thereof, or monomeric or dimeric antigen binding proteins as provided herein or as used to practice methods or other embodiments as provided herein can comprise any “mimetic” and/or “peptidomimetic” form. In alternative embodiments, peptides and polypeptides used to practice embodiments as provided herein can comprise synthetic chemical compounds which have substantially the same structural and/or functional characteristics of the natural polypeptide, for example, a recombinant antibody as provided herein. The mimetic used to practice embodiments as provided herein can be either entirely composed of synthetic, non-natural analogues of amino acids, or, is a chimeric molecule of partly natural peptide amino acids and partly non-natural analogs of amino acids. The mimetic can also incorporate any amount of natural amino acid substitutions, for example, conservative amino acid substitutions, as long as such substitutions also do not substantially alter the mimetic's structure and/or activity. Routine experimentation will determine whether a mimetic is effective for practicing embodiments as provided herein, for example, if a mimetic composition is effective in specifically binding a human CD10 protein. Methodologies detailed herein and others known to persons skilled in the art may be used to select or guide one to choose effective mimetic for practicing the compositions and/or methods as provided herein.

Polypeptide mimetic compositions for practicing embodiments as provided herein can comprise any combination of non-natural structural components. In alternative aspects, mimetic compositions for practicing embodiments as provided herein can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, for example, a beta turn, gamma turn, beta sheet, alpha helix conformation, and the like. For example, a polypeptide can be characterized as a mimetic when all or some of its residues are joined by chemical means other than natural peptide bonds.

In alternative embodiments, an exemplary immunoglobulin heavy chain variable region and/or light claim region comprises an amino acid sequence based on a sequence as set forth in SEQ ID NO:1 or SEQ ID NO:2, respectively, but having one or a plurality of amino acid residue deletions or substitutions, wherein optionally all or some of the amino acid substitutions are conservative amino acid substitutions, and wherein the amino acid sequence (or, the variant polypeptide) with the one or several deletions or substitutions at least substantially retains the ability to specifically bind to human CD10 (neprilysin), albeit the specific binding of the variant can have a binding affinity higher or lower than an antibody or antigen binding fragment thereof, or monomeric or dimeric ABP, as provided herein. In alternative embodiments, the variant polypeptide has between one and about 50 amino acid residue deletions or substitutions, wherein optionally all or some of the amino acid substitutions are conservative amino acid substitutions. In alternative embodiments, the variant polypeptide has between about 1 to 5, 5 to 10, 10 to 15 or 15 to 20 amino acid residue deletions or substitutions.

In alternative embodiments, an exemplary immunoglobulin heavy chain variable region comprises an amino acid sequence as set forth in SEQ ID NO:1 having one, two, three, four, five, six, seven, eight, nine or ten, or between about 1 and 50, amino acid substitutions or deletions, wherein optionally all or some of the substitutions are conservative amino acid substitutions, and retaining the ability to substantially specifically bind to human CD10 (neprilysin).

In alternative embodiments, an exemplary immunoglobulin light chain variable region comprises an amino acid sequence as set forth in SEQ ID NO:2 having one, two, three, four, five, six, seven, eight, nine or ten, or between about 1 and 50, amino acid substitutions or deletions, wherein optionally all or some of the substitutions are conservative amino acid substitutions, and retaining the ability to substantially specifically bind to human CD10 (neprilysin).

Conservative amino acid substitutions are those that substitute a given amino acid in a polypeptide by another amino acid of like characteristics. In alternative embodiments conservative substitutions are the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. In alternative embodiments, other variants are those in which one or more of the amino acid residues of a polypeptide of the invention includes a substituent group. Non-limiting examples of amino acids which may be substituted for an original amino acid in a protein and which may be regarded as conservative substitutions if there is little to no impact on the activity of the polypeptide include: Ala substituted with ser or thr; arg substituted with gln, his, or lys; asn substituted with glu, gln, lys, his, asp; asp substituted with asn, glu, or gln; cys substituted with ser or ala; gln substituted with asn, glu, lys, his, asp, or arg; glu substituted with asn, gln lys, or asp; gly substituted with pro; his substituted with asn, lys, gln, arg, tyr; ile substituted with leu, met, val, phe; leu substituted with ile, met, val, phe; lys substituted with asn, glu, gln, his, arg; met substituted with ile, leu, val, phe; phe substituted with trp, tyr, met, ile, or leu; ser substituted with thr, ala; thr substituted with ser or ala; trp substituted with phe, tyr; tyr substituted with his, phe, or trp; and val substituted with met, ile, leu.

### Identification of Nucleic Acid or Amino Acid Sequences by Sequence Identity

In alternative embodiments, provided are isolated or purified antibodies (Ab), or antigen (Ag) binding fragments thereof, or monomeric or dimeric antigen binding proteins (ABP), having an immunoglobulin heavy chain variable region comprising amino acid sequences having at least about 60%, 65%, 70%, 75%, 80%, 85%, 90%, 95%, 98% sequence identity (or between about 60% to 99% sequence identity) to each of the three CDR1, CDR2 and CDR3 complementarity determining regions (CDRs) of SEQ ID NO:1; or, an amino acid sequence having at least about 60%, 65%, 70%, 75%, 80%, 85%, 90%, 95%, 98% sequence identity (or between about 60% to 99% sequence identity) to SEQ ID NO:1, or an amino acid sequence having complete sequence identity to SEQ ID NO:1.

In alternative embodiments, provided are isolated or purified antibodies (Ab), or antigen (Ag) binding fragments thereof, or monomeric or dimeric antigen binding proteins (ABP), having an immunoglobulin light chain variable region comprising amino acid sequences having at least about 60%, 65%, 70%, 75%, 80%, 85%, 90%, 95%, 98% sequence identity (or between about 60% to 99% sequence identity) to each of the three CDR1, CDR2 and CDR3 complementarity determining regions (CDRs) of SEQ ID NO:2; or, an amino acid sequence having at least about 60%, 65%, 70%, 75%, 80%, 85%, 90%, 95%, 98% sequence identity (or between about 60% to 99% sequence identity) to SEQ ID NO:2, or an amino acid sequence having complete (100%) sequence identity to SEQ ID NO:2.

In alternative embodiments, “percent (%) nucleic acid or amino acid sequence identity” with respect to a reference nucleic acid or polypeptide sequence is defined as the percentage of nucleic acid or amino acid residues in a candidate sequence that are identical with the nucleic acid or amino acid residues in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as GAP™ (Genetics Computer Group, University of Wisconsin, Madison, Wis.) (see for example, Devereux et al., Nucl. Acid. Res., 12:387 (1984), BLASTP™, BLASTN™, BLAST™ BLAST-2™, WU-BLAST2/BLAST v2.0 (see for example, Altschul et al. (1996) Methods Enzymol. 266, 460-480), FASTA™ (see for example, Altschul et al., J. Mol. Biol. (1990) 215:403-410), ALIGN™ (Genentech), MEGALIGN™ (DNASTAR) software, or software for executing the Smith-Waterman algorithm or the Needleman-Wunsch algorithm. Those skilled in the art can determine appropriate parameters for aligning sequences, including any algorithms needed to achieve maximal alignment over the full length of the sequences being compared.

In alternative embodiments, align methods comprise use of a BLAST™ analysis employing: (i) a scoring matrix (such as, e.g., BLOSSUM 62™ or PAM 120™) to assign a weighted homology value to each residue and (ii) a filtering program(s) (such as SEG™ or XNU™) that recognizes and eliminates highly repeated sequences from the calculation. In alternative embodiments, align methods comprise use of a BLAST™ analysis employing a BLAST version 2.2.2 algorithm where a filtering setting is set to blastall-p blastp-d “nr pataa”-F F, and all other options are set to default.

In alternative embodiments, alignment schemes for aligning nucleic acid or two amino acid sequences may result in the matching of only a short region of the two sequences, and this small aligned region may have very high sequence identity even though there is no significant relationship between the two full-length sequences. Thus, alternative embodiments, an exemplary alignment method comprises use of the GAP program, which can result in an alignment that spans at least 50 contiguous amino acids of the target polypeptide. For example, using the computer algorithm GAP™, two polypeptides for which the percent sequence identity is to be determined are aligned for optimal matching of their respective amino acids (the “matched span”, as determined by the algorithm). In certain embodiments, a gap opening penalty (which is calculated as 3 times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250™ or BLOSUM 62™ are used in conjunction with the algorithm. In alternative embodiments, a standard comparison matrix (see for example Dayhoff et al., Atlas of Protein Sequence and Structure, 5(3)(1978) for the PAM 250 comparison matrix; Henikoff et al., Proc. Natl. Acad. Sci USA, 89:10915-10919 (1992) for the BLOSUM 62™ comparison matrix) is also used by the algorithm. In alternative embodiments, the parameters for a polypeptide sequence comparison comprise the following: Algorithm: Needleman et al., J. Mol. Biol., 48:443-453 (1970); Comparison matrix: BLOSUM 62™ from Henikoff et al., supra (1992); Gap Penalty: 12; Gap Length Penalty: 4; Threshold of Similarity: 0. In alternative embodiments, the GAP™ program is used with the above parameters. In certain embodiments, the aforementioned parameters are the default parameters for polypeptide comparisons (along with no penalty for end gaps) using the GAP™ algorithm.

### Purification and Isolation of Antibody Proteins

In alternative embodiments, antibodies, antigen binding fragments thereof, or monomeric or dimeric antigen binding proteins as provided herein (including synthetic or recombinant forms thereof), are substantially purified or isolated, and optionally the substantially purified or isolated forms are the forms used in immunohistochemistry (IHC) methodologies and/or as reagents, kits and/or products of manufacture as provided herein.

In alternative embodiments, antibodies, antigen binding fragments thereof, or monomeric or dimeric antigen binding proteins as provided herein (including synthetic or recombinant forms thereof) are substantially purified or isolated using: physicochemical fractionation, for example, using differential precipitation, size-exclusion or solid-phase binding of immunoglobulins based on size, charge or other shared chemical characteristics of antibodies in typical samples; class-specific affinity, for example, solid-phase binding of particular antibody classes (for example, IgG or IgM) by immobilized biological ligands (for example, proteins, lectins, and the like) that have specific affinity to immunoglobulins, and this can purify all antibodies of the target class without regard to antigen specificity; or antigen-specific affinity, for example, affinity purification of only those antibodies in a sample that bind to a particular antigen molecule through their specific antigen-binding domains, where this purifies all antibodies that bind the antigen without regard to antibody class or isotype.

In alternative embodiments, antibodies, antigen binding fragments thereof, or monomeric or dimeric antigen binding proteins as provided herein (including for example synthetic or recombinant forms), are substantially purified or isolated using standard isolation methodologies such as chromatography, for example, Ion Exchange (IEX) Chromatography, Hydrophobic Interaction Chromatography (HIC), countercurrent chromatography, immunoaffinity and/or size exclusion chromatography.

In alternative embodiments, antibodies, antigen binding fragments thereof, or monomeric or dimeric antigen binding proteins as provided herein (including for example synthetic or recombinant forms) are generated in bioreactors, e.g., a perfusion bioreactor, using continuous expression and purification processes, e.g., as described by Vogg et al Methods Mol Biol. 2018; vol 1850:147-178, or using stirred-tank or rocking bioreactor systems, followed by purification.

### Immunohistochemistry

In alternative embodiments, immunohistochemistry methodologies and/or reagents used with the antibodies (Ab), or antigen binding fragments thereof, or monomeric or dimeric antigen binding proteins as provided herein (including for example synthetic or recombinant forms), products of manufacture, kits or methods as provided herein can include or comprise or comprise use of any IHC protocol, IHC armamentarium, device and/or image or data analysis system, for practicing IHC or IHC reagents known in the art, for example, as described in U.S. patent nos. (USPNs) 10,634,590 (describing a slide holder assembly fixture for use in IHC); 10,565,479 (describing methods for identifying blurred areas in digital images of stained tissue); 10,564,076 (describing systems for analytical (or IHC) sample preparation); 10,551,395 (describing an automated histological staining system); 10,551,378 (describing a tissue staining method); 10,504,224 (describing a digital tissue image analysis system for IHC); 10,501,777 (describing simultaneous, multiplexed detection and quantification of protein expression in IHC); 10,488,340 (describing method for extracting an image of a target fluorophore in a biological material); 10,453,195 (describing methods of detecting tissue areas of interest using digital pathology imaging); 10,438,381 (describing devices, systems and methods for generating a digital image of a tissue section); 10,430,943 (describing methods and programs for automated nuclei area/number estimation for IHC image analysis); 10,416,176 (describing methods for processing specimens in an automated histological staining system); 10,393,633 (describing methods for processing and inhibiting the degradation of an IHC sample); 10,217,011 (describing handling of IHC slides); 10,209,165 (describing automated or semi-automated methods for assessing the quality of staining of a specimen containing cells); 10,126,216 (describing methods for fixing tissue samples for IHC); U.S. Pat. Nos. 9,423,322; 8,515,683 (describing methods and systems for automated detection of immunohistochemical (IHC) patterns); U.S. Pat. No. 10,816,443 (describing automated batch stainers for staining biological specimens on microscope slides); or, U.S. patent application publication nos: US 2019/0178867 A1 (describing detection of specific tissue objects within thin sections of tissue samples as imaged in a brightfield microscope without using a chromogenic stain that is specific to those tissue objects); US 2019/0156510 A1 (describing an image analysis method for analyzing an IHC tissue sample); US 2019/0293637 A1 (methods and systems for quantitative immunohistochemistry (IHC) of a target protein molecule); US 2019/0080450 A1 (describing an automated determination of the staining quality of an IHC stained biological sample); or, US 2020/0316589 A1 (describing a multi-well solid support vessel for the processing and testing of fixed biological materials).

In alternative embodiments, the antibodies, antigen binding fragments thereof, or monomeric or dimeric antigen binding proteins as provided herein (including for example synthetic or recombinant forms) used in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, and IHC protocol or reagents.

In alternative embodiments, methods as provided herein comprise use of chromogenic immunohistochemistry (CIH), wherein a primary antibody (for example, a recombinant antibody (Ab), or antigen binding fragment thereof, or monomeric or dimeric antigen binding protein, as provided herein) or secondary antibody (for example, where the secondary antibody binds to the primary antibody, or the recombinant antibody (Ab), or antigen binding fragment thereof, or monomeric or dimeric antigen binding protein as provided herein,) is conjugated to an enzyme such as peroxidase, for example, an immunoperoxidase), for example, a horseradish peroxidase (HRP), that can catalyze a color-producing reaction. In alternative embodiments, a chromogenic moiety used in methods as provided herein is or comprises a coumarin; a rhodamine; 2,3,6,7-tetrahydro-11-oxo-1H,5H,11H-[1]benzopyrano[6,7,8-ij]quinolizine-1-0-carboxylic acid; 7-(diethylamino)coumarin-3-carboxylic acid; a coumarin derivative; a rhodamine derivative; a tetramethylrhodamine; a diarylrhodamine derivative; QSY 7; QSY 9; QSY 21; diazo chromophores; DABSYL; tartrazine; triarylmethane compounds; fast red; fast blue; fuchsin; Cascade Blue acetyl; Dapoxylsulfonic acid/carboxylic acid succinimidyl ester; DY-405; Alexa Fluor 405 succinimidyl ester; Cascade Yellow succinimidyl ester; pyridyloxazole succinimidyl ester (PyMPO); Pacific Blue succinimidyl ester; DY-415; 7-hydroxycoumarin-3-carboxylic acid succinimidyl ester; DYQ-425; 6-FAM phosphoramidite; Lucifer Yellow; iodoacetamide; Alexa Fluor 430 succinimidyl ester; Dabcyl succinimidyl ester; NBD chloride/fluoride; QSY 35 succinimidyl ester; DY-485XL; Cy2 succinimidyl ester; DY-490; Oregon Green 488 carboxylic acid succinimidyl ester; Alexa Fluor 488 succinimidyl ester; BODIPY 493/503 C3 succinimidyl ester; DY-480XL; BODIPY FL C3 succinimidyl ester; BODIPY FL C5 succinimidyl ester; BODIPY FL-X succinimidyl ester; DYQ-505; Oregon Green 514 carboxylic acid succinimidyl ester; DY-510XL; DY-481XL; 6-carboxy-4′,5′-dichloro-2′,7′-dimethoxyfluorescein succinimidyl ester (JOE); DY-520XL; DY-521XL; BODIPY R6G C3 succinimidyl ester; erythrosin isothiocyanate; 5-carboxy-2′,4′,5′,7′-tetrabromosulfonefluorescein succinimidyl ester; Alexa Fluor 532 succinimidyl ester; 6-carboxy-2′,4,4′,5′7,7′-hexachlorofluorescein succinimidyl ester (HEX); BODIPY 530/550 C3 succinimidyl ester; DY-530; BODIPY TMR-X succinimidyl ester; DY-555; DYQ-1; DY-556; Cy3 succinimidyl ester; DY-547; DY-549; DY-550; Alexa Fluor 555 succinimidyl ester; Alexa Fluor 546 succinimidyl ester; DY-548; BODIPY 558/568 C3 succinimidyl ester; Rhodamine red-X succinimidyl ester; QSY 7 succinimidyl ester; BODIPY 564/570 C3 succinimidyl ester; BODIPY 576/589 C3 succinimidyl ester; carboxy-X-rhodamine (ROX); succinimidyl ester; Alexa Fluor 568 succinimidyl ester; DY-590; BODIPY 581/591 C3 succinimidyl ester; DY-591; BODIPY TR-X succinimidyl ester; Alexa Fluor 594 succinimidyl ester; DY-594; carboxynaphthofluorescein succinimidyl ester; DY-605; DY-610; Alexa Fluor 610 succinimidyl ester; DY-615; BODIPY 630/650-X succinimidyl ester; erioglaucine; Alexa Fluor 633 succinimidyl ester; Alexa Fluor 635 succinimidyl ester; DY-634; DY-630; DY-631; DY-632; DY-633; DYQ-2; DY-636; BODIPY 650/665-X succinimidyl ester; DY-635; Cy5 succinimidyl ester; Alexa Fluor 647 succinimidyl ester; DY-647; DY-648; DY-650; DY-654; DY-652; DY-649; DY-651; DYQ-660; DYQ-661; Alexa Fluor 660 succinimidyl ester; Cy5.5 succinimidyl ester; DY-677; DY-675; DY-676; DY-678; Alexa Fluor 680 succinimidyl ester; DY-679; DY-680; DY-682; DY-681; DYQ-3; DYQ-700; Alexa Fluor 700 succinimidyl ester; DY-703; DY-701; DY-704; DY-700; DY-730; DY-731; DY-732; DY-734; DY-750; Cy7 succinimidyl ester; DY-749; DYQ-4; Cy7.5 succinimidyl ester; 7-diethylaminocoumarin-3-carboxylic acid; succinimidyl ester; Dabsyl sulfonyl chloride; fluorescein isothiocyanate (FITC) carboxy succinimidyl ester (DY-495); Rhodamine Green carboxylic acid succinimidyl ester (DY-505); eosin isothiocyanate (EITC); 6-carboxy-2′,4,7,7′-tetrachlorofluorescein succinimidyl ester (TET); carboxyrhodamine 6G succinimidyl ester; carboxytetramethylrhodamine succinimidyl ester (TMR, TAMRA) (DY-554); QSY 9 succinimidyl ester; sulforhodamine B sulfonyl chloride (DY-560); Texas Red (sulforhodamine 101); gallocyanine; Fast Green FCF; Malachite Green; or, a QSY 21 succinimidyl ester.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors, or a fluorophore or dye as described in U.S. patent application no. US 2019/0018018 A1. 3,3′-Diaminobenzidine (DAB) also can be used.

In alternative embodiments, methods as provided herein comprise use of a direct method or one-step staining method where a primary antibody (for example, antibodies (Ab), or antigen binding fragments thereof, or monomeric or dimeric antigen binding proteins as provided herein (including for example synthetic or recombinant forms)) is labeled and reacts directly with an antigen, for example, in a tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, CD10), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sectioned or sliced or used whole. Before sectioning, the tissue sample can be embedded in a medium, for example, paraffin wax or cryomedia. Tissue sections can be sectioned or sliced on a variety of instruments, most commonly using a microtome, cryostat, or vibratome. Specimens can be sectioned or sliced at a range of about 3 μm to 5 μm. The sections or slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make a CD10 epitope available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain System) (Agilent, San Jose, CA), which allows for staining of two or more markers on a single slide. In alternative embodiments, the IHC is performed using an EnVision FLEX HRP Magenta, High pH (Dako Omnis) system, and binding can be visualized by EnVision FLEX HRP Magenta Chromogen. In alternative embodiments, the IHC is performed using EnVision FLEX Mini Kit, High pH, which is a high-sensitivity visualization system intended for use in IHC together with Dako AUTOSTAINER™ instruments; this dual link system detects primary mouse and rabbit antibodies and the reaction is visualized by 3,3′-Diaminobenzidine (DAB) chromogen (DAB forms a water-insoluble brown precipitate when oxidized, for example, by a peroxidase).

### Products of Manufacture and Kits

Provided are products of manufacture and kits comprising anti-human CD10 Abs as provided herein (including for example synthetic or recombinant forms), and for practicing methods as provided herein using anti-human CD10 Abs as provided herein (including for example synthetic or recombinant forms); and optionally the products of manufacture and/or kits can further comprise some or all reagents needed to perform an immunohistochemistry (IHC), and optionally can comprise instructions for practicing methods as provided herein.

In alternative embodiment, products of manufacture have attached thereto or affixed (optionally covalently bound) on or onto an antibody or a dimeric antigen binding protein as provided herein (including for example synthetic or recombinant forms), and optionally products of manufacture as provided herein are or comprise arrays, chips, biochips, slides, trays, dishes (for example, microtiter dishes), phages or phagemids.

In alternative embodiment, immunohistochemistry methodologies and/or reagents used to practice composition, product of manufacture (for example, kit) or method embodiments as provided herein can include or comprise or comprise use of any IHC protocol, IHC armamentarium, device and/or image or data analysis system, for practicing IHC or IHC reagents known in the art, for example, as described in U.S. patent nos. 10,565,479 (describing methods for identifying blurred areas in digital images of stained tissue); 10,564,076 (describing systems for analytical (or IHC) sample preparation); 10,551,395 (describing an automated histological staining system); 10,551,378 (describing a tissue staining method); 10,504,224 (describing a digital tissue image analysis system for IHC); 10,501,777 (describing simultaneous, multiplexed detection and quantification of protein expression in IHC); 10,488,340 (describing method for extracting an image of a target fluorophore in a biological material); 10,453,195 (describing methods of detecting tissue areas of interest using digital pathology imaging); 10,438,381 (describing devices, systems and methods for generating a digital image of a tissue section); 10,416,176 (describing methods for processing specimens in an automated histological staining system); 10,393,633 (describing methods for processing and inhibiting the degradation of an IHC sample); 10,217,011 (describing handling of IHC slides); 10,209,165 (describing automated or semi-automated methods for assessing the quality of staining of a specimen containing cells); 10,126,216 (describing methods for fixing tissue samples for IHC); 9,423,322.

In alternative embodiment, products of manufacture and kits can further comprise a human CD10 polypeptide, which can have the amino acid sequence (see for example Strausberg et al Proc Natl Acad Sci USA (2002) vol 99(26):16899-16903):

```
(SEQ ID NO: 3)
    1 mgksesqmdi tdintpkpkk kqrwtpleis lsvlvlllti iavtmialya tyddgickss
  61 dciksaarli qnmdattepc tdffkyacgg wlkrnvipet ssrygnfdil rdelevvlkd
121 vlqepktedi vavqkakaly rscinesaid srggepllkl lpdiygwpva tenweqkyga
181 swtaekaiaq Inskygkkvl inlfvgtddk nsvnhvihid qprlglpsrd yyectgiyke
241 actayvdfmi svarlirqee rlpidenqla lemnkvmele keianatakp edrndpmlly
301 nkmtlaqiqn nfsleingkp fswlnftnei mstvnisitn eedvvvyape yltklkpilt
361 kysardlqnl mswrfimdlv sslsrtykes rnafrkalyg ttsetatwrr canyvngnme
421 navgrlyvea afageskhvv edliaqirev fiqtlddltw mdaetkkrae ekalaikeri
481 gypddivsnd nklnneylel nykedeyfen iiqnlkfsqs kqlkklrekv dkdewisgaa
541 vvnafyssgr nqivfpagil qppffsaqqs nslnyggigm vigheithgf ddngrnfnkd
601 gdlvdwwtqq sasnfkeqsq cmvyqygnfs wdlaggqhln gintlgenia dngglgqayr
661 ayqnyikkng eekllpgldl nhkqlfflnf aqvwcgtyrp eyavnsiktd vhspgnfrii
721 gtlqnsaefs eafhcrknsy mnpekkcrvw
```

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Development of Exemplary Anti-Human CD10 Antibodies

This example describes the developments of antibodies as provided herein and their superior efficacy in IHC assays.

An antigenic peptide from the extracellular domain of human CD10 (neprilysin) was designed. The peptide was conjugated to keyhole limpet hemocyanin (KLH) and used for immunization of 10 mice. Following 3 immunizations blood was drawn from each mouse to obtain plasma samples. The plasma was tested in ELISA and subsequently in IHC to determine immune response in the individual mice.

5 mice were evaluated in group 1 (gives specific IHC staining), 3 mice were in group 2 (gives specific IHC staining, but also non-specific staining), and 1 mouse in group 4 (no specific staining). The last mouse died before testing.

Mice were chosen for generation of hybridomas and were sacrificed to harvest the spleen. Hybridoma fusion was performed according to traditional methods using the myeloma fusion cell line P3X63Ag8 (ATCC® TIB-9™).

From fusion #481, performed on a mix of 2 mice, 17 clones were positive in ELISA. Of these 6 clones were evaluated to be in group 1, 3 in group 2 and the remaining 8 in group 4 (no specific staining). Of the 6 clones in group 1 the 2 best performers, 7D5 and 10A4 were subcloned. The best performing clone F461, 7D5, H8 was subcloned and tested on more tissues.

A further subcloning was performed on F461, 7D5, H8, B7 together with 2 other promising clones (from different mother clones). Final clone is F461, 7D5, H8, B7, G8. (Isotype IgGI).

A cell bank was created in form of Original Cell Stocks (OCS). Test of the cell bank was performed by taking a vial of OCS and placing into culture for test of bank, mycoplasma tasting and production of antibody in from of OCS Supernatant (OCSS). This OCSS were used for further testing, and the resultant exemplary antibody was named Mab3124.

Exemplary antibody Mab3124 has a heavy chain comprising SEQ ID NO:1 and a light chain comprising SEQ ID NO:2.

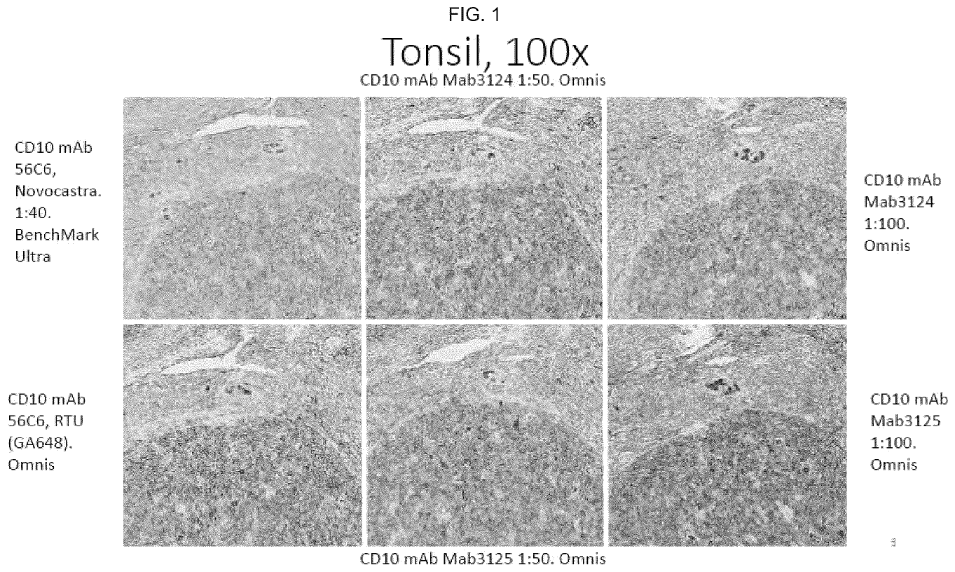

Testing of the specificity and optimal performance of this antibody was made on multiple tissues, and the exemplary Mab3124 antibody was compared to the golden standard of CD10 for IHC, the mouse monoclonal antibody 56C6 (ABCAM, Burlingame, CA), as illustrated in FIG. 1 to FIG. 15, which used the protocol:
Heat-Induced Epitope Retrieval (HIER) in Target Retrieval Solution (TRS) (Agilent, Dako, Santa Clara, CA) high pH (3-in-1), 30 min.
Primary Ab incubation: 20 min.
Detection system: EnVision FLEX™ (Agilent, Dako, Santa Clara, CA), 20 min. in polymer.
Stainer: OMNIS™ automated staining solution (Agilent, Dako, Santa Clara, CA),

The IHC images of FIG. 1 to FIG. 15, comparing the IHC imaging capabilities of the mouse monoclonal antibody 56C6 (ABCAM, Burlingame, CA) versus the exemplary antibody Mab3124 clearly show the superior CD10 binding capabilities of the exemplary antibody Mab3124 when used in IHC:

FIG. 1 illustrates an IHC 100× image of tonsil tissue sample;

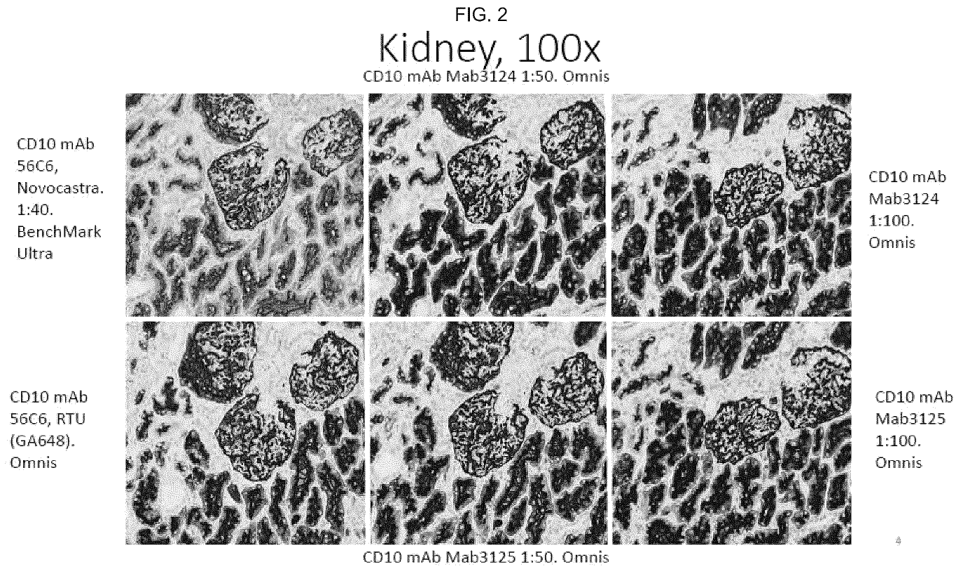

FIG. 2 illustrates an IHC 100× image of kidney tissue sample;

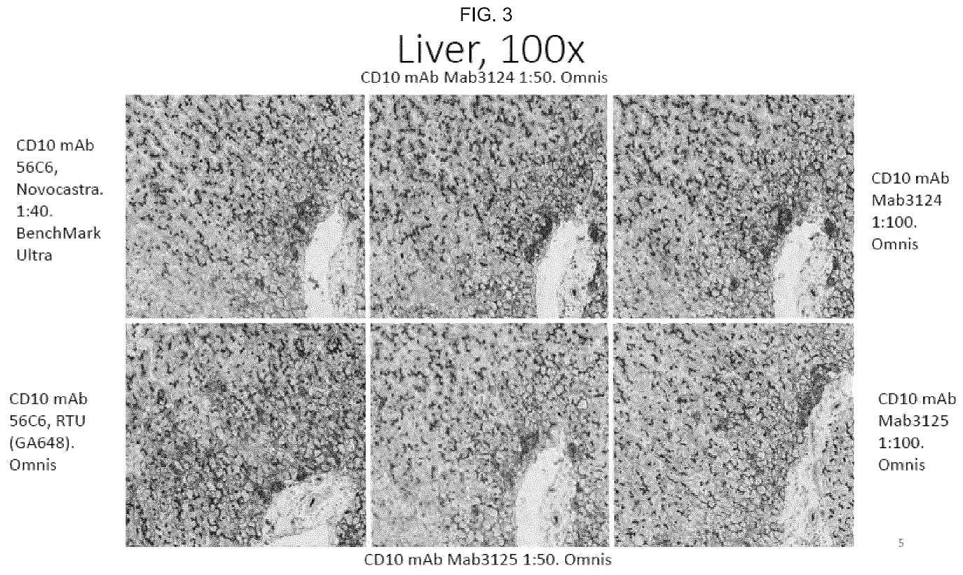

FIG. 3 illustrates an IHC 100× image of liver tissue sample;

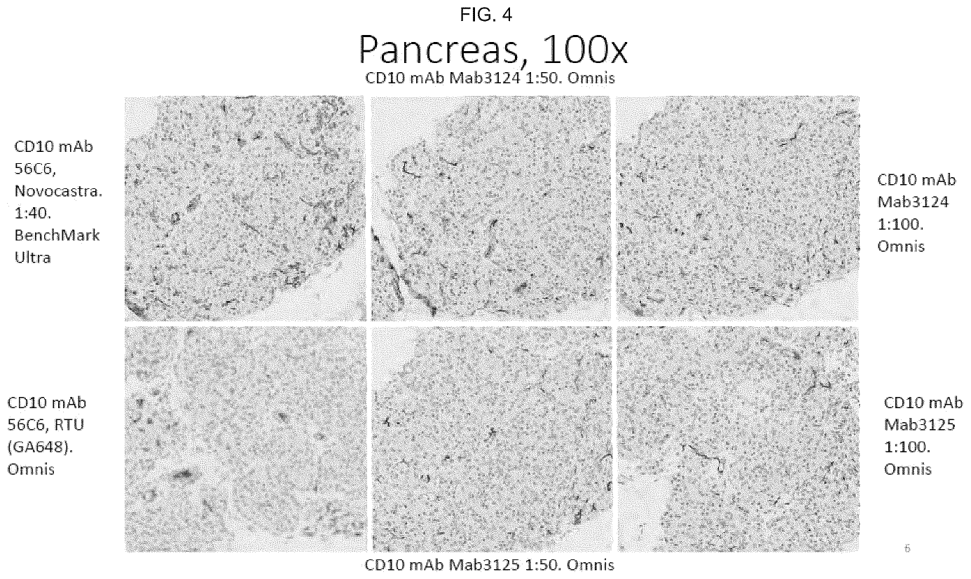

FIG. 4 illustrates an IHC 100× image of pancreas tissue sample;

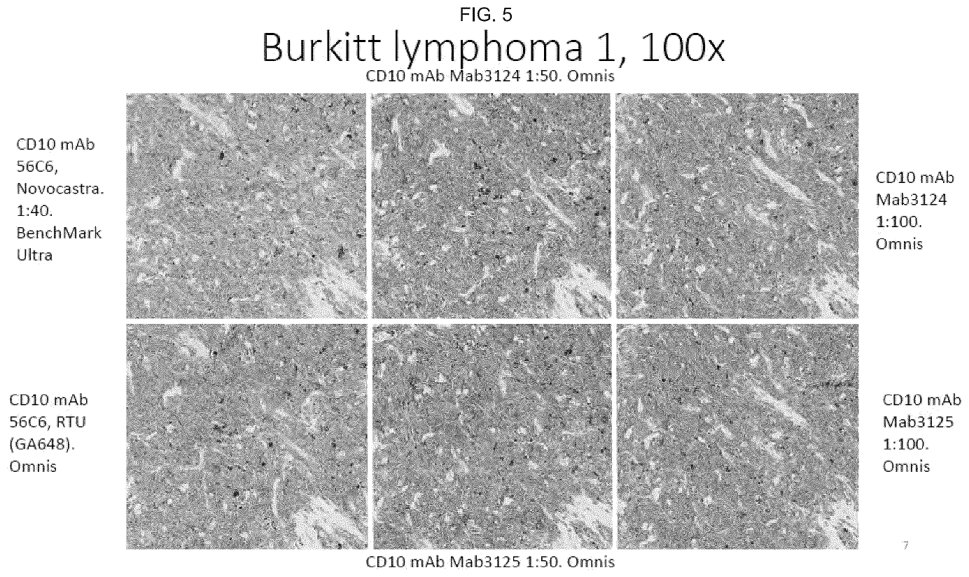

FIG. 5 illustrates an IHC 100× image of a Burkitt's lymphoma tissue section;

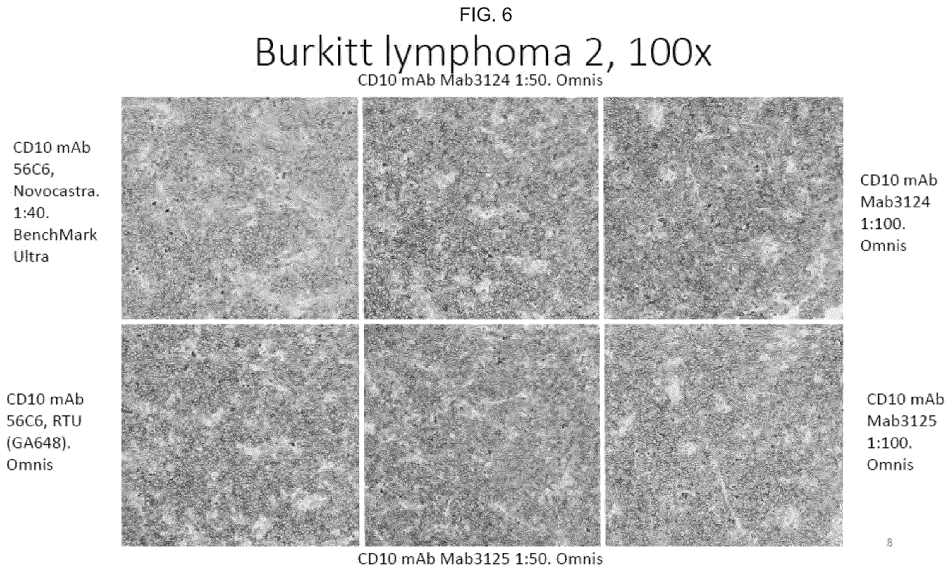

FIG. 6 illustrates an IHC 100× image of a Burkitt's lymphoma tissue section;

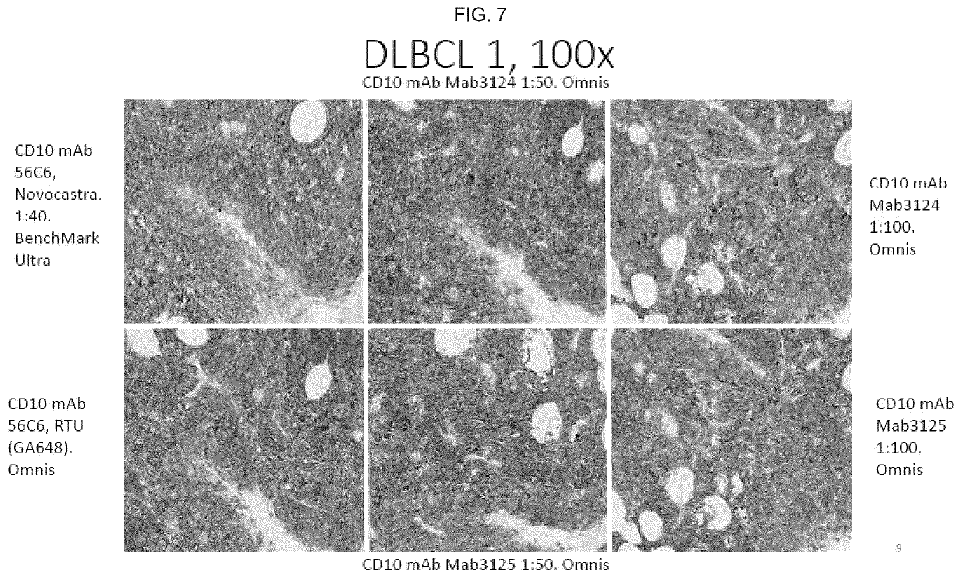

FIG. 7 illustrates an IHC 100× image of a diffuse large B-cell lymphoma (DLBCL) tissue sample;

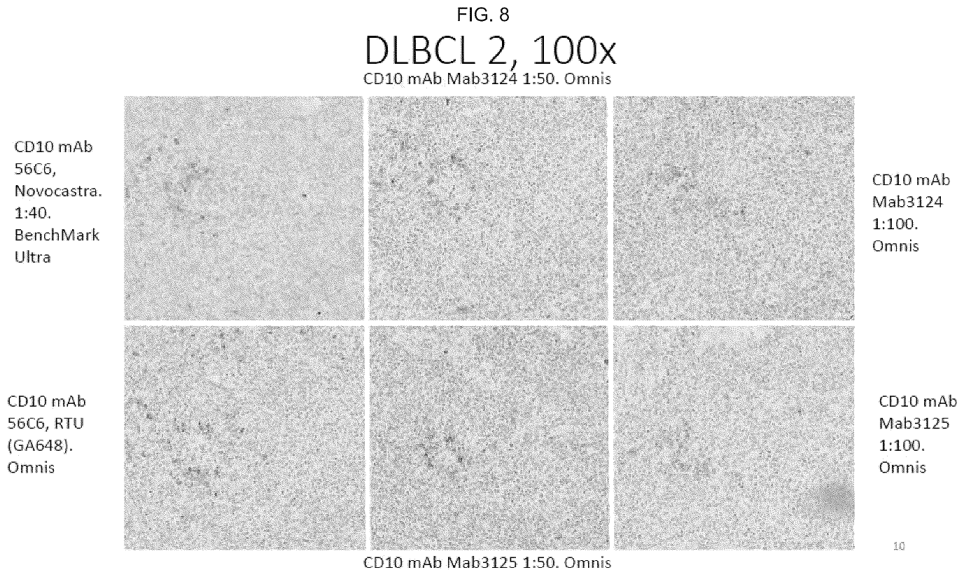

FIG. 8 illustrates an IHC 100× image of a DLBCL tissue sample;

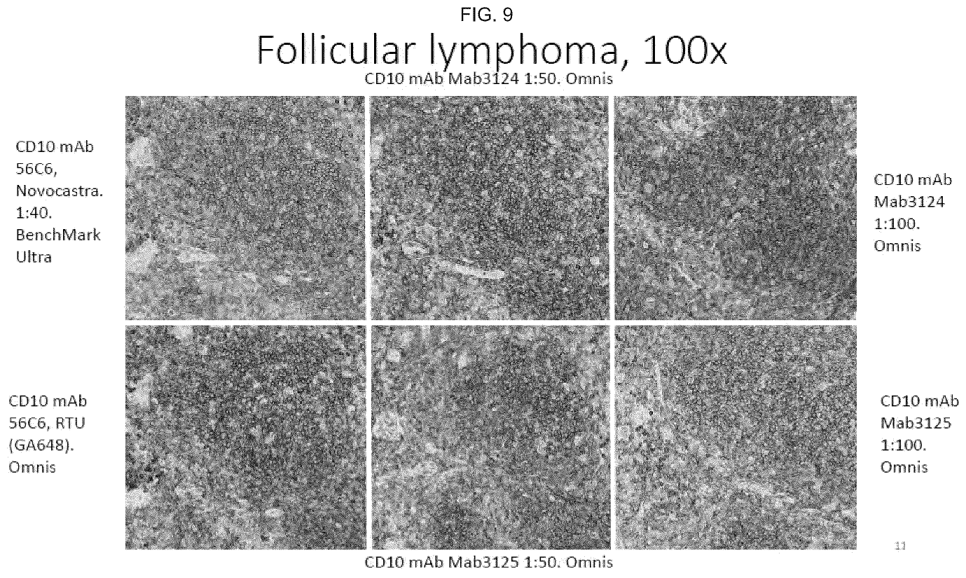

FIG. 9 illustrates an IHC 100× image of a follicular lymphoma sample;

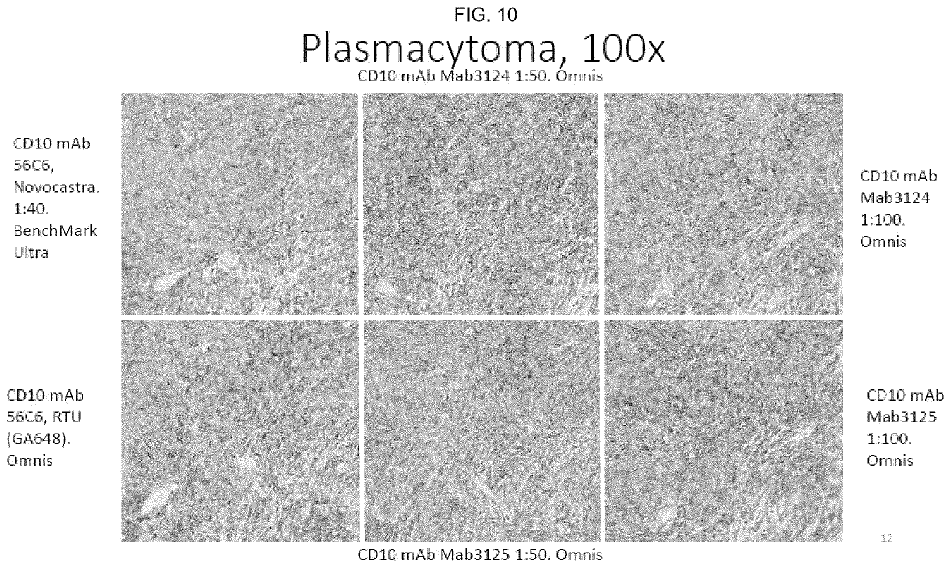

FIG. 10 illustrates an IHC 100× image of a plasmacytoma sample;

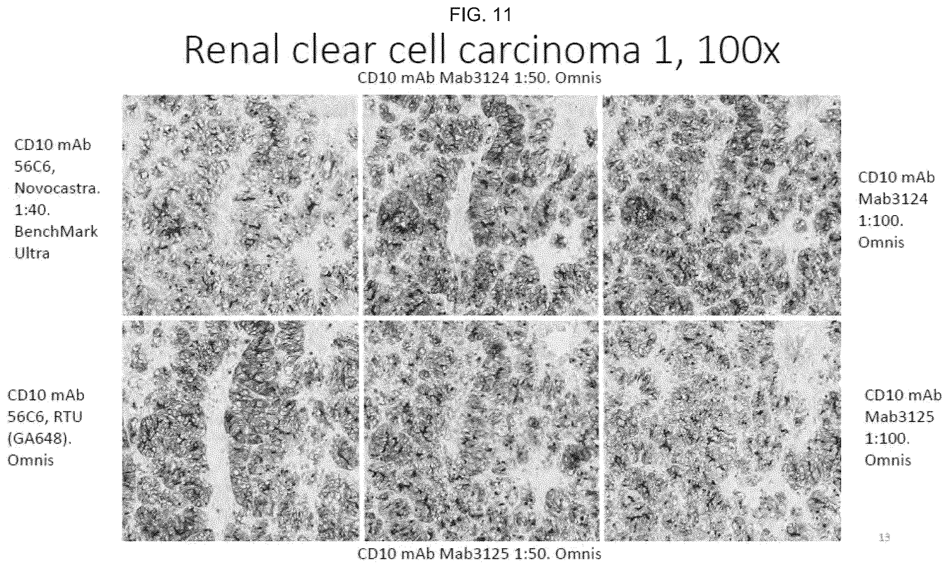

FIG. 11 illustrates an IHC 100× image of a renal clear cell carcinoma sample;

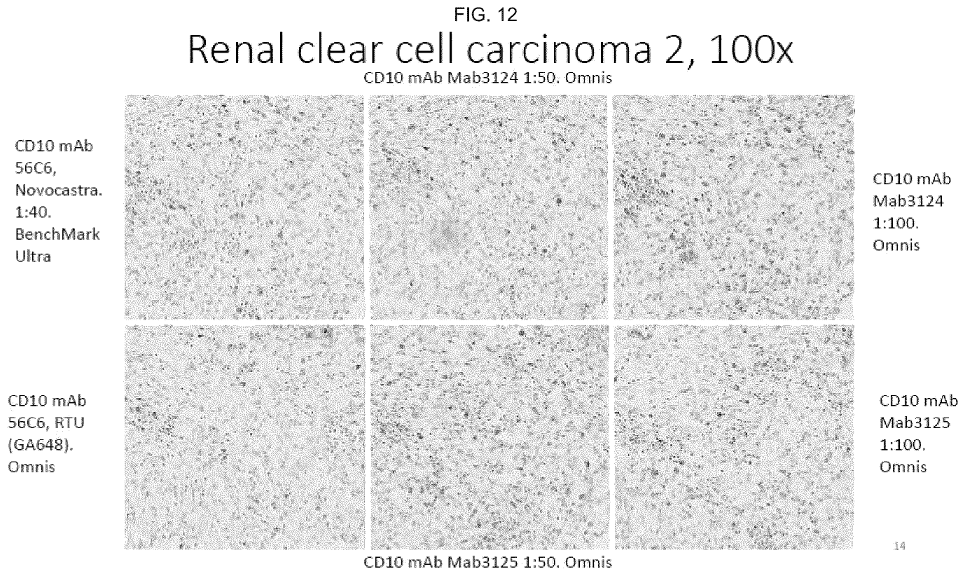

FIG. 12 illustrates an IHC 100× image of a renal clear cell carcinoma sample;

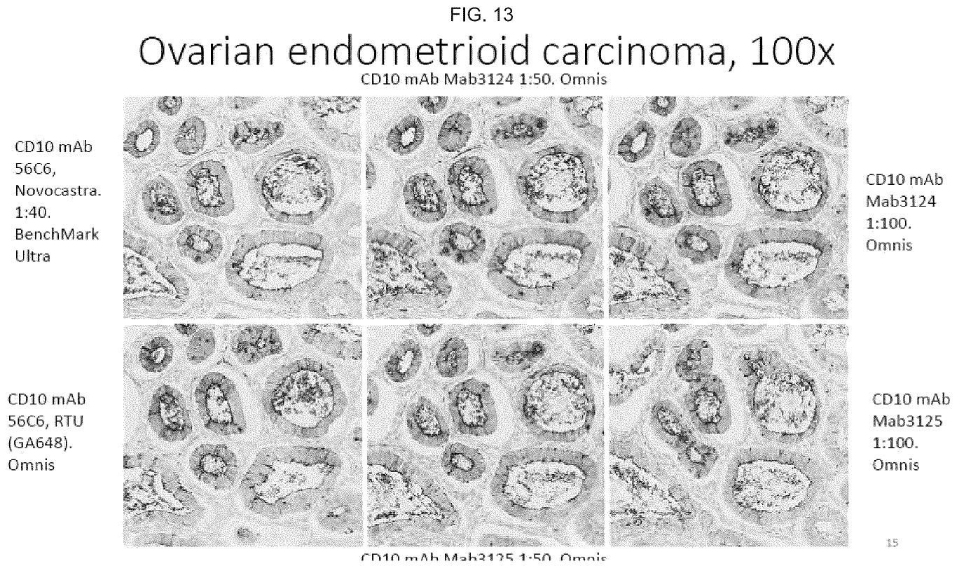

FIG. 13 illustrates an IHC 100× image of an ovarian endometrioid carcinoma sample;

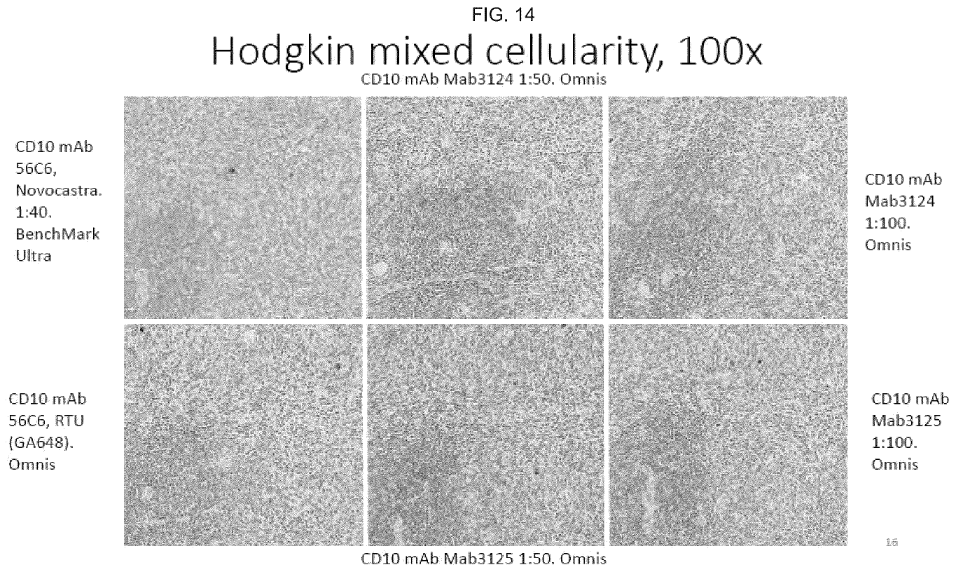

FIG. 14 illustrates an IHC 100× image of a Hodgkin's mixed cellularity tissue sample;

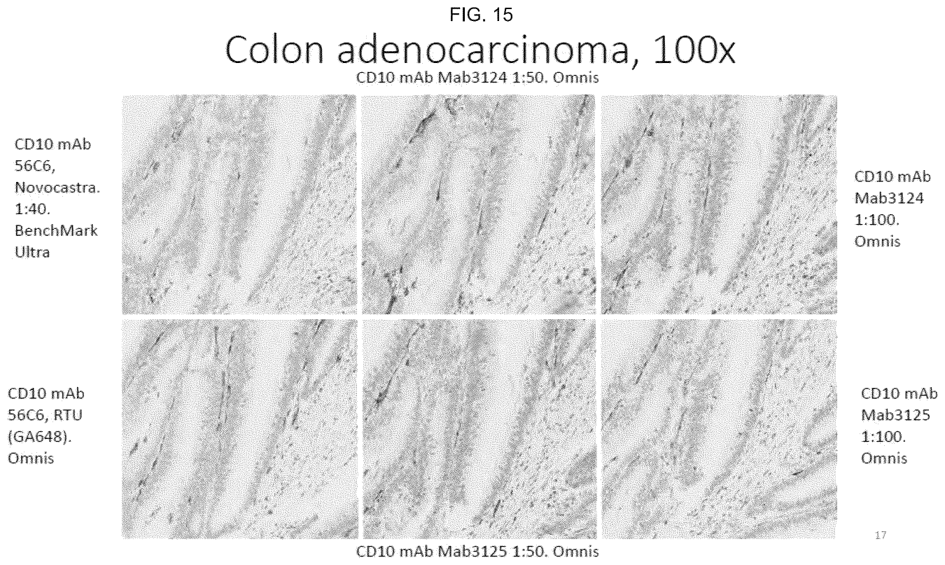

FIG. 15 illustrates an IHC 100× image of a colon adenocarcinoma issue sample.

The antibody F461, 7D5, H8, B7, G8 (also called Mab 3124) obtained best possible score. The sensitivity of the newly developed clone shows higher sensitivity than the golden standard (antibody 56C6) and was used without amplification in a simple FLEX reagent (Agilent, Dako, Santa Clara, CA) protocol (no linker).

### Normal CD10 Tissue Specificity:

In liver, bile canaliculi show a moderate to strong staining reaction. In tonsil, germinal center B-cells show a weak to moderate staining reaction. The antibody label stems cells in the bone marrow, a subset of immature B cells in bone marrow and neutrofiles. The antibody also labels renal glomerular and proximal tubular cells, germinal centers in lamina propria of the colon, brush borders of the enterocytes in the small intestine, gallbladder brush border epithelium, interstitial stromal cells of the lung, Schwann nerve cells, interfascicular stromal cells in striated muscles, fibroblasts, syncytial trophoblasts and cytotrophoblasts of the placenta, prostate glandular epithelium, stromal cells in the endometrium and breast myoepithelial cells.

The staining pattern should be membranous but also cytoplasmic.

Negative tissues/structures include mantle zone B-cells and T-cells in the tonsil. Distal tubular cells in the kidney.

### B-Cell Lymphomas:

In general, CD10 positive expression among lymphomas do not confirm that a specific lymphoma is in fact a B-cell lymphoma. CD10 is part of a panel of antibodies (B-cell markers), which positive expression may indicate B-cell lymphoma. Some B-cell lymphomas may have lost their CD10 receptors and these will not express CD10. Therefor any of the B-cell lymphomas described below may also be included as negative clinical tissues.

### Follicular Lymphomas

Most Follicular Lymphoma (FL grade IIIB may not) express CD10 with a moderate to strong membraneous/cytoplasmic staining reaction. FL may consist of poorly defined neoplastic follicles with an attenuated or absent mantle zone. Diffuse areas lacking follicles may be present and the neoplastic cells may spread to the interfollicular areas. Two different staining patterns and intensities may be seen representing large centroblastic cells in the follicular areas and the extended B-cell zone in the interfollicular area. CD10 expression is often stronger in the follicles than in the interfollicular neoplastic cells and may be absent in the interfollicular component. Some normal cells may be present in FL, namely Follicular Dendritic cells, follicular T-cells and different kind of histiocytes.

### Burkitt Lymphoma

Burkitt Lymphoma consist of medium size tumor cells with a diffuse monotonous growth pattern and the CD10 expression is a usually, strong homogenous membraneous/cytoplasmic staining reaction. “Starry sky” pattern is usually present, representing numerous benign macrophages that have ingested apoptotic tumor cells.

### Diffuse Large B-Cell Lymphoma

Diffuse Large B-cell Lymphoma DLBCL, Not Otherwise Specified, is a neoplasm of large B lymphoid cells with nuclear size equal to or larger than a normal macrophage nuclei or more than twice the size of a normal lymphocyte.

Morphological DLBCL can be categorized into centroblastic variant, immunoblastic variant and anaplastic variant. The centroblastic variant has medium-sized to large lymphoid cells with oval/round vesicular nuclei containing fine chromatin and two to four nuclear membrane-bound nucleoli. The immunoblastic variant consist of immunoblasts with s single centrally located nucleolus and an appreciable amount of basophilic cytoplasma. The anaplastic variant is characterized by large to very large round, oval or polygonal cells with bizarre pleomorphic nuclei. CD10 is expressed in 30-50% of these tumors. CD10 may help to subclassify DLBCL, NOS into germinal center-like and non-germinal center-like.

### Renal Cell Carcinoma:

The most common morphological type of RCC is the clear cell type, which consists of large cells with distinct cell membranes and centrally placed small nuclei. The cytoplasma is clear and vacuolized. The clear cell carcinoma is typically CD10 positive, while collecting duct carcinoma, another type of RCC, are negative. The diagnostic use of CD10 in renal cell carcinoma also includes differentiation of the rare metastasis from other organs to the kidney, and identification of renal metastasis to other organs (15).

### Negative Clinical Tissues:

Periferal T-cell lymphoma; Marginal zone lymphoma (this type of lymphoma may be CD10 neg or CD10 positive); Mantle cell lymphoma; Burkitt lymphoma (this type of lymphoma may be CD10 neg or CD10 positive); DLBCL (this type of lymphoma may be CD10 neg or CD10 positive); Follicular lymphoma (this type of lymphoma may be CD10 neg or CD 10 positive); Non-clear cell Renal cell lymphoma; Renal cell carcinoma, metastatic from other organs.

Another series of antibody IHC tests identified a preferred protocol for the antibodies and subsequent evaluation of the immunohistochemical performance:

| Antibody/Clone/ | 1. | Mouse monoclonal anti-human CD10/Mab3124/IgG1 |
|---|---|---|
| Ig-type | 2. | Mouse monoclonal anti-human CD10/Mab3125/IgG1 |
| Reference Ab and | 3: | CD10 mAb clone 56C6 (ready to use (RTU)), GA648). HIER: |
| protocol | | high pH 30 min., Primary Ab incubation: 12½ min., Blocking: 3 |
| | | min., Link: 10 min., Polymer: 20 min., Cromogen: 5 min. |
| | | Performed on OMNIS ™ (Dako, Agilent, Santa Clara, CA) |
| | 4: | CD10 mAb clone 56C6 1:40 (Novocastra, NCL-L-CD10-270), 32 |
| | | min., HIER by CC1 pH 8.5 48 min. at 99° C., OptiView as |
| | | detection system and performed on Ultra, VMS. |
| Test material | 1. | TMA Normal |
| All fixed | 2. | TMA Neoplasia |
| in 10% NBF | 3. | NQC CD10 run 39: Tonsil, Kidney, Renal |
| | | clear cell carcinoma, Burkitt lymphoma, |
| | 4. | TMA Lymphoma 1 |
| | 5. | TMA Lymphoma 2 |
| HIER conditions | 1. | HIER in Target Retrieval Solution High pH (3-in-1), 30 |
| tested | | min. |
| Recommended | 2. | HIER in Target Retrieval Solution High pH (3-in-1), 30 |
| | | min. |
| Titers tested | 1: | 1:50/100/200 - incubation 20 min. |
| | 2: | 1:50/100/200 - incubation 20 min. |
| Recommended | 1: | 1:100 - incubation 20 min. |
| | 2: | 1:50 - incubation 20 min. |
| Diluent | Dako antibody diluent K8006 | |
| Detection syst. | 1. | EnVision FLEX+, linker 10 min., polymer 20 |
| tested | | min./EnVision Flex, polymer 20 min. |
| Recommended | 2. | EnVision FLEX+, linker 10 min., polymer 20 |
| | | min./EnVision Flex, polymer 20 min. |

| | |
|---|---|
| Immunostainer | OMNIS ™ (Dako, Agilent, Santa Clara, CA) |

| | | Pos. | Pos. | Pos. | Pos. |
|---|---|---|---|---|---|
| | | mAb Mab3124 | mAb Mab3125 | mAb 56C6 | mAb 56C6 |
| | | high pH, | high pH, | high pH, | high pH, |
| Tumor | N | 1:100 Dako | 1:50 Dako | RTU Dako | 1:40 Novocastra |
| Ductal breast carcinoma | 2 | 2+ | 2+ | 2+ | 2+ |
| Lobular breast carcinoma | 1 | 1− | 1− | 1− | 1− |
| Colon adenocarcinoma | 2 | 2− | 2− | 2− | 2− |
| Urothelial carcinoma | 2 | 2− | 2− | 2− | 2− |
| Pancreatic adenocarcinoma | 2 | 2− | 2− | 2− | 2− |
| Lung adenocarcinoma | 2 | 2− | 2− | 2− | 2− |
| Lung squamous cell carcinoma | 1 | 1− | 1− | 1− | 1− |
| Thyroid follicular carcinoma | 1 | 1− | 1− | 1− | 1− |
| Thyroid medullary carcinoma | 1 | 1− | 1− | 1− | 1− |
| Serous ovarian carcinoma | 2 | 2− | 2− | 2− | 2− |
| Ovarian clear cell carcinoma | 1 | 1− | 1− | 1− | 1− |
| Ovarian endometrioid carc. | 1 | 1+ | 1+ | 1+ | 1+ |
| Uterine endometrioid carc. | 1 | 1− | 1− | 1− | 1− |
| Uterine endocervical ad. carc. | 1 | 1− | 1− | 1− | 1− |
| Prostate adenocarcinoma | 2 | 1−, 1+ | 1−, 1+ | 1−, 1+ | 1−, 1+ |
| Renal clear cell carcinoma | 3 | 1−, 2+ | 1−, 2+ | 1−, 2+ | 1−, 2+ |
| Intestinal carcinoid | 1 | 1− | 1− | 1− | 1− |
| Seminoma | 2 | 2+ | 2+ | 2+ | 2+ |
| Melanoma | 1 | 1− | 1− | 1− | 1− |
| GIST | 1 | 1− | 1− | 1− | 1− |
| Leiomyosarcoma | 1 | 1− | 1− | 1− | 1− |
| Rhabdomyosarcoma | 1 | 1+ | 1+ | 1+ | 1+ |
| Hodgkin mixed cellularity | 6 | 6− | 6− | 6− | 6− |
| Hodgkin lymphocyte-depleted | 5 | 5− | 5− | 5− | 5− |
| Hodgkin nodular lymphocyte | 5 | 5− | 5− | 5− | 5− |
| Hodgkin nodular sclerosis | 2 | 2− | 2− | 2− | 2− |
| T-cell lymphoma, peripheral | 3 | 3− | 3− | 3− | 3− |
| T-cell lymphoma, anapl. | 1 | 1− | 1− | 1− | 1− |
| Lymphoplasmacytic lymphoma | 6 | 6− | 6− | 6− | 6− |
| Burkitt lymphoma | 5 | 5+ | 5+ | 5+ | 5+ |
| Plasmacytoma | 5 | 5− | 5− | 5− | 5− |
| Extraosseous plasmacytoma | 5 | 5+ | 5+ | 5+ | 5+ |
| B-CLL | 6 | 6− | 6− | 6− | 6− |
| DLBCL | 7 | 5−, 2+ | 5−, 2+ | 5−, 2+ | 5−, 2+ |
| Mantle cell lymphoma | 6 | 6− | 6− | 6− | 6− |
| Follicular lymphoma | 8 | 8+ | 8+ | 8+ | 8+ |

### Discussion

### Reference mAb Clone 56C6

mAb clone 56C6, as ready to use (RTU) (Dako, Agilent, Santa Clara, CA) and concentrate from Novocastra, Leica Biosystems (Buffalo Grove, IL) was used as reference for the two exemplary CD10 antibodies tested. The protocols were performed on the OMNIS™ (Dako, Agilent, Santa Clara, CA) and BENCHMARK ULTRA™ (Ventana, Roche Diagnostics) using the protocols listed above.

In general, the concentrated Ab from Novocastra gave a weaker staining intensity than the RTU Ab from Dako. For both references, a weak to moderate membranous staining reaction was seen in virtual all germinal centre B-cells in tonsil, a moderate staining reaction in bile canaliculi in liver and a strong membranous and cytoplasmic staining reaction in epithelial cells of the proximal tubules in the kidney. The RTU Ab from Dako obtained the best signal to noise ratio.

In the neoplasias, the staining profiles were as listed above. 5/5 Burkitt lymphomas and ⅔ renal clear cells were positive using a cut-off of greater than or equal to (≥) 10%. All 18 tested Hodgkin lymphomas were negative.

### Exemplary mAb Clone Mab3124:

Using the mentioned test parameters, the best protocol with shortest turnaround time (TAT) and best signal-to-noise ratio was a primary Ab titer of 1:100, HIER in Target Retrieval Solution (TRS) (Agilent, Dako, Santa Clara, CA), high pH and ENVISION FLEX™ (EnVision Flex) (Agilent, Santa Clara, CA) as detection system.

Reducing the titer, for example, 1:200, provided a slightly reduced sensitivity, especially in the germinal center B-cells in tonsil and was not recommended for final evaluation. When using a titer of 1:50 a faint background were seen in a few tissues.

Using the selected settings, the following results for the exemplary mAb clone Mab3124 were obtained: generally, the staining pattern in both proportion of positive cells and staining intensity was similar to the reference mAb 56C6 as RTU from Dako when using the selected protocol for the exemplary mAb clone Mab3124. In all germinal centre B-cells in tonsil a weak to moderate distinct membranous staining reaction was seen. A strong cytoplasmic and membranous staining reactions was obtained in epithelial cells of the proximal tubules in the kidney.

The staining profile in the neoplasias were similar to the reference Abs (see table).

### mAb Clone Mab3125:

In this test, using the mentioned test parameters, the best protocol with shortest TAT and best signal-to-noise ratio was a primary Ab titer of 1:50, HIER in Target Retrieval Solution (TRS) (Agilent, Dako, Santa Clara, CA) High pH and ENVISION FLEX™ (EnVision Flex) (Agilent, Santa Clara, CA) as detection system.

The staining result was for the mAb clone Mab3125 virtually identical to both the reference RTU Ab from Dako and the exemplary mAb clone Mab3124 in the normal tissues.

### Conclusion:

In this test, performed with the protocol and settings listed and material selected, as discussed above, the best overall result was obtained by the exemplary mAb clone Mab3124. The result was comparable to the reference based on mAb 56C6 as RTU stained on OMNIS™ (Dako, Agilent, Santa Clara, CA).

CD10 mAb Mab3125 gave almost the same result as both the reference and the mAb Mab3124, however the exemplary Mab3124 provided a slightly stronger staining intensity

The staining pattern for the neoplasias were similar to both the reference Abs and the exemplary mAb clone Mab3124.

### Example 2: IHC Protocols Using the Exemplary mAb Clone Mab3124

The IHC performance of the new exemplary CD10 Mab 3124 was tested on AUTOSTAINER LINK™; and we compared the IHC performance of the exemplary CD10 Mab 3124 using AUTOSTAINER LINK™ (Dako, Agilent, Santa Clara, CA) to the exemplary CD10 Mab 3124 using OMNIS™ (Dako, Agilent, Santa Clara, CA). The premise of the test was that CD10 Mab 3124 using AUTOSTAINER LINK™ ready-to-use should be identical to CD10 Mab 3124 using OMNIS™ ready-to-use.

An Autostainer Link compatibility test for CD10 (Mab 3124) that the IHC protocol developed for CD10 with OMNIS™ also was compatible with AUTOSTAINER LINK™. The IHC protocol tested in the AUTOSTAINER LINK™ compatibility test for CD10 Mab 3124 was Target Retrieval Solution (TRS) (Agilent, Dako, Santa Clara, CA) high pH, ENVISION FLEX™ 20 minutes incubation, and Mab 3124 concentration of 4 μg/ml (diluted in 50809™, a ready to use antibody diluent (Daco, Agilent, Santa Clara, CA)) 20 minutes incubation.

The performance of CD10 clone Mab 3124 on AUTOSTAINER LINK™ in a IHC protocol very similar to the protocol developed for CD 10 Mab 3124 on OMNIS™ Optimal protocol for CD 10 Mab 3124 on AUTOSTAINER LINK™ is shown in the table below; note that this protocol is very similar to the protocol developed for CD 10 Mab 3124 using OMNIS™.

| | AUTOSTAINER LINK ™ | OMNIS ™ |
|---|---|---|
| | Protocol for | protocol for |
| | CD10 (Mab 3124), | CD10 (Mab 3124), |
| | Clone F461, 7D5, | Clone F461, 7D5, |
| Reagent | H8, B7, G8 | H8, B7, G8 |
| Target Retrieval | High pH | High pH |
| 1° Ab Diluent | S0809 ™ (Dako, Agilent) | S0809 ™ (Dako, |
| | | Agilent) |
| 1° Ab dilution and | 4.0 μg/ml in 20 minutes | 4.0 ug/mL/20 mins |
| concentration/inc. time | | |
| Peroxidase blocking | 5 minutes | 3 mins |
| reagent | | |
| Linker? | No | No |
| Visualization | FLEX ™/High | FLEX ™/High/ |
| system/inc. time | 20 minutes | 20/20 |
| Chromogen | EnVision FLEX ™ DAB+ | EnVision FLEX ™ |
| | 2 * 5 minutes | DAB+ 5 mins |

The table below summarizes the staining protocol for CD10 Mab 3124 on AUTOSTAINER LINK™. “Ab 1×” corresponds to CD10 Mab 3124 diluted in S0809™ to a concentration of 4 μg/ml.

| Code | Reagent name | Volume (μl) | Incubation |
|---|---|---|---|
| | Buffer | | 0 |
| SM801 | FLEX Peroxidase Block | 150 | 5 |
| | Buffer | | 0 |
| | Ab 1x | 150 | 20 |
| | Buffer | | 0 |
| SM802 | FLEX/HRP | 150 | 20 |
| | Buffer | | 0 |
| | Buffer | | 5 |
| SM803 | FLEX DAB+ Sub-Chromo | 150 | 5 |
| SM803 | FLEX DAB+ Sub-Chromo | 150 | 5 |
| | Buffer | | 0 |
| SM806 | FLEX Hematoxylin | 150 | 5 |
| | DI Water | | 0 |
| | Buffer | | 5 |
| | DI Water | | 0 |

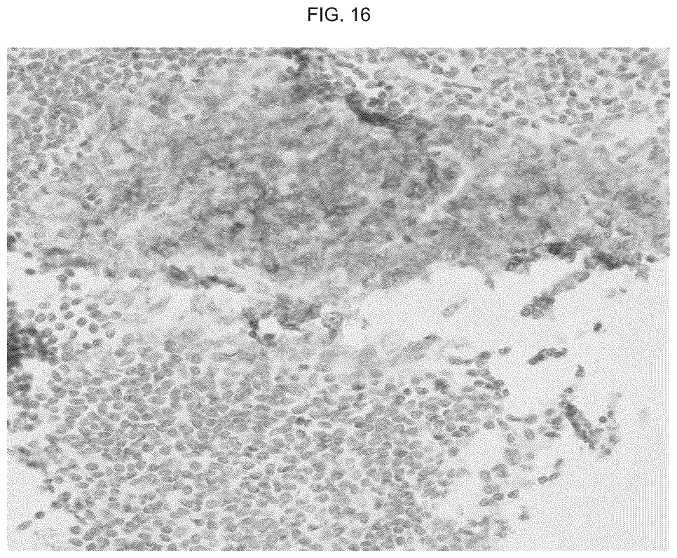

Optimal protocol for CD10 Mab 3124 on AUTOSTAINER LINK™ was applied on various multi-tissue arrays (TMA 1 lymfom, TMA 2 lymfom and CD10 NordiQC run 39). CD10 Mab 3124 on AUTOSTAINER LINK™ was assessed. FIG. 16 to FIG. 20 illustrates images shows IHC staining of CD10 tissue slides with CD10 Mab 3124, optimal protocol (1× at approximately 4 μg/ml) on AUTOSTAINER LINK™:

FIG. 16 illustrates an image of tonsil tissue stained with CD10 Mab 3124, using optimal protocol (1× at approximately 4 μg/ml) on AUTOSTAINER LINK™; Note that germinal center B-cells show a moderate to strong and membrane staining, and note that only a single germinal center is present in this tonsil specimen.

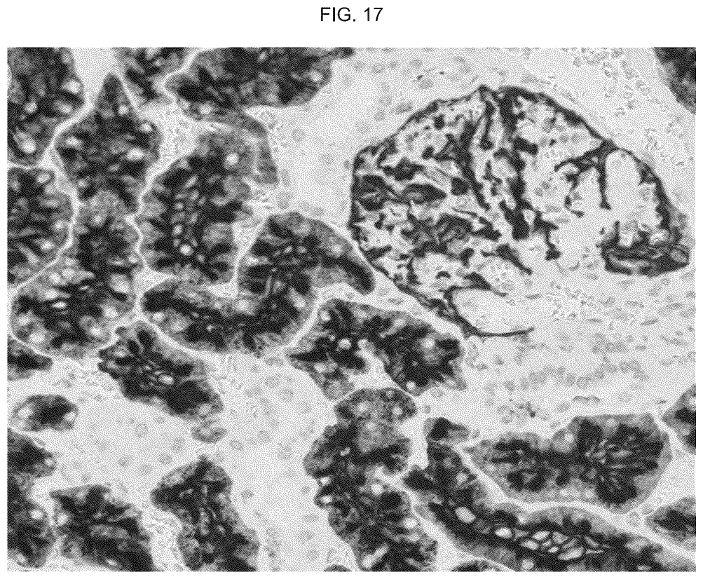

FIG. 17 illustrates an image of kidney tissue stained with CD10 Mab 3124, using optimal protocol (1× at approximately 4 μg/ml) on AUTOSTAINER LINK™; Note that the epithelial cells of the proximal tubules and glomeruli in kidney show an intense membrane and cytoplasmic staining.

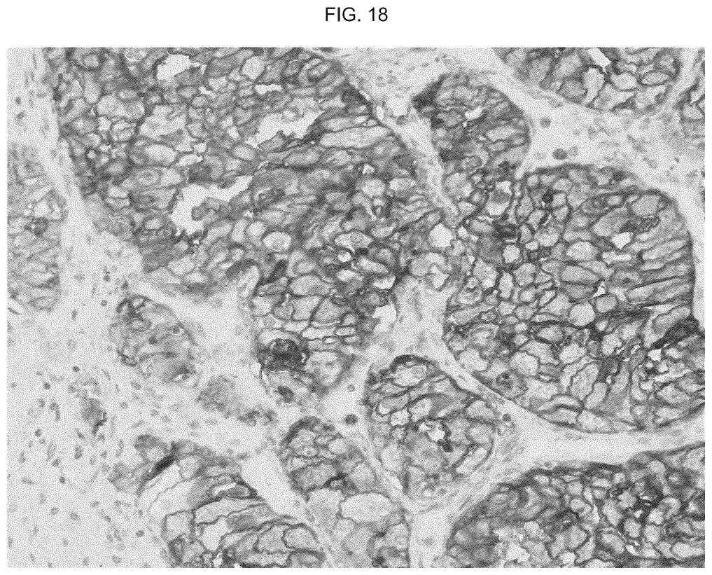

FIG. 18 illustrates an image of a renal clear cell carcinoma stained with CD10 Mab 3124, using optimal protocol (1× at approximately 4 μg/ml) on AUTOSTAINER LINK™; note that neoplastic cells of renal clear cell carcinoma show a moderate/strong and distinct membrane staining;

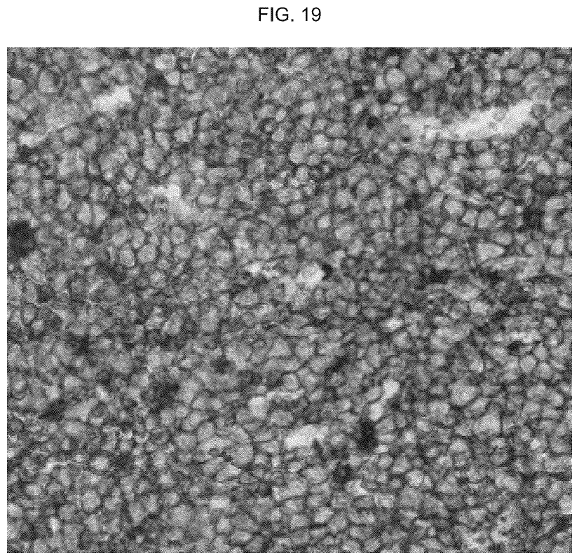

FIG. 19 illustrates an image of Burkitt lymphoma stained with CD10 Mab 3124, using optimal protocol (1× at approximately 4 μg/ml) on AUTOSTAINER LINK™ Note that neoplastic cells of Burkitt lymphoma show a moderate/strong and distinct membrane staining;

FIG. 20 illustrates an image of tonsil tissue stained with CD10 Mab 3124, using optimal protocol (1× at approximately 4 μg/ml) on AUTOSTAINER LINK™; Note that neoplastic cells of follicular lymphoma show a moderate/strong and distinct membrane staining.

### CD10 Mab 3124 on AUTOSTAINER LINK™ Versus CD10 Mab 3124 on OMNIS™

Performance of CD10 Mab 3124 on AUTOSTAINER LINK™ IX (approximately 4 μg/ml) compared to CD10 Mab 3124 on OMNIS™, 1× (approximately 4 μg/ml) is shown in the table below.

Average staining intensity for CD10 Mab 3124 CX on positive tissues was 2.474 for CD10 Mab 3124 on AUTOSTAINERLINK™ 4× and 2.388 for CD10 Mab 3124 on OMNIS™.

Furthermore, application of double concentration ½× (approximately 8 μg/ml) of CD10 Mab 3124 on AUTOSTAINER LINK™ didn't change the status of negative clinical tissues.

No background staining was observed when CD10 Mab 3124 was replaced with NCR in the staining protocol for CD10 on AUTOSTAINER LINK™.

The staining intensity of CD 10 Mab 3124 AUTOSTAINER LINK™ 1× and CD10 Mab 3124 on OMNIS™ 1× on various tissues types are shown in the table below:

| | | | CD10 | IR750 | CD10 | CD10 |
|---|---|---|---|---|---|---|
| | | | (Mab 3124) | (NCR to | (Mab 3124) | (Mab 3124) |
| | | | (4 μg/ml) | CD10 | (8 μg/ml) | (4 μg/ml) |
| | | | (~1x) | (Mab 3124) | (~0.5X) | (~1X) |
| Block | | | (AS Link) | (AS Link) | (AS Link) | Omnis |
| # | Tissue | Disease | AF0458-18 | AF0459-18 | AF0460-18 | AF0461-18 |
| 61877 | Liver | Lg multi | 2.625 | 0 | | 2.875 |
| | (HE1) | block | | | | |
| 61877 | Tonsil | Lg multi | 1.875 | 0 | | 2.25 |
| | (LE1) | block | | | | |
| 61877 | Mamma | Lg multi | 1.625 | 0 | | 1.75 |
| | carcinoma | block | | | | |
| | (CT1) | | | | | |
| 106731 | Liver | Lg multi | 3 | 0 | | 3.25 |
| | (HE3) | block | | | | |
| 106731 | Tonsil | Lg multi | 2.5 | 0 | | 2.5 |
| | (LE3) | block | | | | |
| 106731 | Mamma | Lg multi | 2 | 0 | | 1.75 |
| | carcinoma | block | | | | |
| | (CT3) | | | | | |
| 110306 | Liver | Lg multi | 3.5 | 0 | | 3.375 |
| | (HE2) | block | | | | |
| 110306 | Tonsil | Lg multi | 2.875 | 0 | | 2.625 |
| | (LE2) | block | | | | |
| 110306 | Mamma | Lg multi | 2.375 | 0 | | 2.25 |
| | carcinoma | block | | | | |
| | (CT2) | | | | | |
| 51392 | Kidney | Renal cell | 2.875 | 0 | | 2.125 |
| | | carcinoma | | | | |
| 51393 | Kidney | Renal cell | 2.625 | 0 | | 2.25 |
| | | carcinoma | | | | |
| 110441 | Lymph | Marginal zone | 3.5 | 0 | | 3.75 |
| | node | lymphoma | | | | |
| 110442 | Lymph | Marginal zone | 3.625 | 0 | | 3.625 |
| | node | lymphoma | | | | |
| 110439 | Liver | Hepatocellular | 3 | 0 | | 2.75 |
| | | carcinoma | | | | |
| 110440 | Liver | Hepatocellular | 2.875 | 0 | | 2.625 |
| | | carcinoma | | | | |
| 103783 | Neck & Inn | Burkitt | 2.875 | 0 | | 2.625 |
| | | lymphoma | | | | |
| 103785 | Neck & Inn | Burkitt | 3 | 0 | | 2.75 |
| | | lymphoma | | | | |
| 110443 | Lymph | Burkitt | 0 | 0 | 0 | 0 |
| | node | lymphoma | | | | |
| 110444 | Lymph | Burkitt | 0 | 0 | 0 | 0 |
| | node | lymphoma | | | | |
| 110446 | Lymph | SLL | 0 | 0 | 0 | 0 |
| | node | | | | | |
| 110447 | Lymph | SLL | 0 | 0 | 0 | 0 |
| | node | | | | | |
| 110450 | Unknown | Marginal zone | 0.125 | 0 | 0.125 | 0.125 |
| | | lymphoma | | | | |
| 110451 | Unknown | Marginal zone | 0.125 | 0 | 0.125 | 0.125 |
| | | lymphoma | | | | |
| 110452 | Lymph | Marginal zone | 0 | 0 | 0 | 0 |
| | node | lymphoma | | | | |
| 110453 | Lymph | Marginal zone | 0 | 0 | 0 | 0 |
| | node | lymphoma | | | | |
| 61330 | Prostrate | SLL | 0 | 0 | 0 | 0 |

| | | |
|---|---|---|
| Average score (positive tissue) | 2.474 | |
| (CD10 (Mab 3124 (AS Link) | | |
| Average score (positive tissue) | | 2.388 |
| (CD10 (Mab 3124 (Omnis) | | |

| | |
|---|---|
| Difference average score (CD10 | 0.086 |
| AS Link) and CD10 (Onmis) | |

A number of embodiments of the invention have been described. Nevertheless, it can be understood that various modifications may be made without departing from the spirit and scope of the invention. Accordingly, other embodiments are within the scope of the following claims.

1: An isolated or purified antibody (Ab), or antigen (Ag) binding fragment thereof, or monomeric or dimeric antigen binding protein (ABP), capable of specifically binding a human CD10 polypeptide,
wherein the isolated or purified Ab, or Ag binding fragment thereof, or monomeric or dimeric ABP comprises:
(a) an immunoglobulin heavy chain variable region comprising:
(i) an amino acid sequence comprising the three CDR1, CDR2 and CDR3 complementarity determining regions (CDRs) of SEQ ID NO:1, or CDR1 amino acid (aa) residues GYTFTDYF (residues 26-33 of SEQ ID NO:1), CDR2 aa residues INPNNGDT (residues 51 to 58 of SEQ ID NO:1), and CDR3 aa residues AKGGFNPGDY (residues 97-106 of SEQ ID NO:1), or
(ii) amino acid sequences having at least about 70%, 75%, 80%, 85%, 90%, 95%, 98% sequence identity to each of the three CDR1, CDR2 and CDR3 complementarity determining regions (CDRs) of SEQ ID NO:1, or CDR1 amino acid (aa) residues GYTFTDYF (residues 26-33 of SEQ ID NO:1), CDR2 aa residues INPNNGDT (residues 51 to 58 of SEQ ID NO:1), and CDR3 aa residues AKGGFNPGDY (residues 97-106 of SEQ ID NO:1), or
(iii) an amino acid sequence having at least about 70%, 75%, 80%, 85%, 90%, 95%, 98% sequence identity to SEQ ID NO:1, or an amino acid sequence having complete sequence identity to SEQ ID NO:1;
(b) an immunoglobulin light chain variable region comprising:
(i) an amino acid sequence comprising the three CDR1, CDR2 and CDR3 complementarity determining regions (CDRs) of SEQ ID NO:2, or CDR1 amino acid (aa) residues QSLVHRNGNTY (residues 27-37 of SEQ ID NO:2), CDR2 aa residues KVS (residues 55 to 57 of SEQ ID NO:2), and CDR3 aa residues SQSTHVPLT (residues 94-102 of SEQ ID NO:2), or
(ii) amino acid sequences having at least about 70%, 75%, 80%, 85%, 90%, 95%, 98% sequence identity to each of the three CDR1, CDR2 and CDR3 complementarity determining regions (CDRs) of SEQ ID NO:2, or CDR1 amino acid (aa) residues QSLVHRNGNTY (residues 27-37 of SEQ ID NO:2), CDR2 aa residues KVS (residues 55 to 57 of SEQ ID NO:2), and CDR3 aa residues SQSTHVPLT (residues 94-102 of SEQ ID NO:2); or
(iii) an amino acid sequence having at least about 70%, 75%, 80%, 85%, 90%, 95%, 98% sequence identity to SEQ ID NO:2, or an amino acid sequence having complete (100%) sequence identity to SEQ ID NO:2; or
(c) the immunoglobulin heavy chain variable region of (a) and the immunoglobulin light chain variable region of (b).

2: The isolated or purified Ab, or Ag binding fragment thereof, or monomeric or dimeric ABP, of claim 1, fabricated as or in the form of:
a Fab, or an Ab fragment having just one constant and one variable domain of each of an Ab heavy and light chain,
a F(ab′)₂ or an Ab digested by pepsin yielding two fragments: a F(ab′)₂ fragment and a pFc′ (pepsin cleavage Fc) fragment,
a Fab′ or a single chain of a F(ab′)₂ fragment,
a single-chain variable fragment (scFv) or a fusion protein of a variable region of an Ab heavy and light chain connected together with a linker peptide optionally of about ten to about 25 amino acids in length,
a (scFv)₂, or a di-scFv or a bi-scFv, or a single peptide chain having two variable heavy and two variable light regions yielding tandem scFv,
a minibody or a fusion protein of a variable region of an Ab heavy and light chain connected together with an alkyl group, optionally a methyl or an ethyl group,
a diabody or an scFv with a linker peptide too short, optionally about five amino acids, for the two variable regions to fold together forcing the scFvs to dimerize), a triabody or a tetrabody, or an scFv with a linker peptide too short, optionally about one or two amino acids, for the two variable regions to fold together forcing the scFvs to trimerize or tetramize,
a single-domain antibody (dAB) or a single variable region of an Ab heavy or Ab light chain,
a plurality of complementarity determining region (CDR) fragments, or
a multispecific antibody formed from two or more antibody fragments.

3: The isolated or purified Ab, or Ag binding fragment thereof, or monomeric or dimeric ABP, of claim 1, wherein the immunoglobulin heavy chain variable region comprises:
an amino acid sequence:
(SEQ ID NO: 1)
EVQLQQSGPDLVKPGASVKMSCKASGYTFTDYFMKWVKQSHGKSLEWIGD
INPNNGDTFYNQKFKGKATLTVDKSSSTAYMQLSSLTSEDSAVYYCAKGG
FNPGDYWGQGTTLTVSS,
SEQ ID NO:1 having one or more amino acid substitutions, additions (insertions) or deletions, and the recombinant Ab, or Ag binding fragment thereof, or monomeric or dimeric ABP retains its ability to specifically bind to a human CD10 protein or polypeptide.

4: The isolated or purified Ab, or Ag binding fragment thereof, or monomeric or dimeric ABP, of claim 3, wherein: the one or more amino acid substitutions comprise one or more conservative amino acid substitutions.

5: The isolated or purified Ab, or Ag binding fragment thereof, or monomeric or dimeric ABP, of claim 1,
wherein the immunoglobulin light chain variable region comprises an amino acid sequence:
(SEQ ID NO: 2)
DAVLTQAPLSLPVSLGDQASISCRSSQSLVHRNGNTYLHWFLQRPGQSPK
LLIDKVSNRFSGVPDRFSGSGSGTDFTLKISRVEAEDLGVYFCSQSTHVP
LTFGAGTKLELK,
SEQ ID NO:2 having one or more amino acid substitutions, additions (insertions) or deletions, and the recombinant Ab, or Ag binding fragment thereof, or monomeric or dimeric ABP retains its ability to specifically bind to a human CD10 protein or polypeptide.

6: The isolated or purified Ab, or Ag binding fragment thereof, or monomeric or dimeric ABP of claim 1, wherein:
(a) the one or more amino acid substitutions comprise one or more conservative amino acid substitutions;
(b) SEQ ID NO:1 or SEQ ID NO:2 has two, three, four, five, six, seven, eight, nine, ten, eleven, twelve thirteen, fourteen or fifteen conservative amino acid substitutions, and the recombinant Ab, or Ag binding fragment thereof, or monomeric or dimeric ABP retains its ability to specifically bind to a human CD10 protein or polypeptide;
(c) the immunoglobulin light chain variable region further comprises at least a portion of a light chain constant region;
(d) the immunoglobulin heavy chain variable region further comprises at least a portion of an immunoglobulin heavy chain constant region;
(e) the immunoglobulin light chain variable region further comprises at least a portion of an immunoglobulin light chain constant region; and, the immunoglobulin heavy chain variable region further comprises at least a portion of an immunoglobulin heavy chain constant region;
(f) the heavy chain constant region comprises amino acid sequence from a IgG, IgM, IgA, IgD or IgE isotype;
(g) the light chain constant region comprises amino acid sequence from a kappa (κ) or lambda (λ) isotype;
(h) the at least a portion of the heavy chain constant region, at least a portion of the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat;
(i) at least a portion of the heavy chain constant region, at least a portion of the light chain constant region, or at least a portion of the heavy chain constant region and the light chain constant region, is or comprises a synthetic amino acid sequence, or
(j) any combination of (a) to (i).

7: The isolated or purified Ab, or Ag binding fragment thereof, or monomeric or dimeric ABP, of claim 1, wherein the recombinant Ab, the Ag binding fragment thereof, or monomeric or dimeric ABP, or the heavy chain constant region, or the light chain constant region, or the heavy chain constant region and the light chain constant region, further comprises or is bound to a heterologous protein, peptide, or a compound or a composition.

8: The isolated or purified Ab, or Ag binding fragment thereof, or monomeric or dimeric ABP, of claim 7, wherein the heterologous protein or peptide, or the compound or a composition, comprises a detectable protein, a detectable agent or a binding moiety.

9: The isolated or purified Ab, or Ag binding fragment thereof, or monomeric or dimeric ABP, of claim 8, wherein the heterologous protein or peptide comprises a carrier protein.

10: The isolated or purified Ab, or Ag binding fragment thereof, or monomeric or dimeric ABP, of claim 7, wherein the heterologous protein, peptide or the compound or composition, is covalently conjugated to the recombinant antibody (Ab), or Ag binding fragment thereof, or monomeric or dimeric ABP.

11: The isolated or purified Ab, or Ag binding fragment thereof, or monomeric or dimeric ABP, of claim 1, wherein:
(a) the detectable agent or binding moiety comprises a biotin, a fluorescent or chemiluminescent label, a fluorophore, perylene, fluorenyl, coumarin, 7-methoxycoumarin (Mca), 4-(dimethylaminoazo)benzene-4-carboxylic acid (dabcyl), Tamra, boron-dipyrromethene (BODIPY), or derivatives thereof, a dye, a radioisotope, a quantum dot or photoluminescent aqueous nanocrystal, a hapten, or an antibody binding epitope or domain;
(b) the dye is or comprises rhodamine, [2-(4-nitro-2,1,3-benzoxadiazol-7-yl)aminoethyl]trimethylammonium (NBD), nile red or nile blue, or is a fluorescent dye comprising sulfoindocyanine;
(c) the fluorophore is or comprises a dansyl, a fluorescein, a carboxyfluorescein (FAM) or a 6-FAM moiety;
(d) the dye is or comprises a cyanine dye, a Cy3 or a Cy5;
(e) the hapten is or comprises a biotin, a theophylline, a digoxigenin, a carborane, a fluorescein or a bromodeoxyuridine moiety;
(f) the Ab, or Ag binding fragment thereof, or monomeric or dimeric ABP is a recombinant Ab, or Ag binding fragment thereof, or monomeric or dimeric ABP, or comprises a peptide or polypeptide made by a recombinant technique; or
(g) any combination of (a) to (f).

12: A chimeric or recombinant nucleic acid comprising: a nucleic acid sequence encoding an Ab, or an Ag binding fragment thereof, or monomeric or dimeric ABP of claim 1,
wherein optionally the chimeric or recombinant nucleic acid further comprises and is operatively linked to a transcriptional regulatory element,
and optionally the transcriptional regulatory element comprises a promoter, and optionally the promoter is an inducible promoter or a constitutive promoter.

13: An expression cassette, a vector, a recombinant virus, an artificial chromosome, a cosmid or a plasmid comprising a chimeric or a recombinant nucleic acid of claim 12.

14: A cell comprising a chimeric or recombinant antibody or dimeric antigen binding protein of claim 1,
wherein the cell is a bacterial, fungal, mammalian, yeast, insect or plant cell, and optionally the mammalian cell is a human cell.

15: A method for detecting the presence of a human CD10 protein in or on a cell, a tissue, an organ or a portion of any of the foregoing, comprising:
(a) contacting the cell, tissue or organ or portion of any of the foregoing with an Ab, or Ag binding fragment thereof, or monomeric or dimeric ABP of claim 1, and
(b) detecting the specific binding of the Ab, or Ag binding fragment thereof, or monomeric or dimeric ABP with a CD10 polypeptide, or a polypeptide or peptide comprising an antigen or an epitope consisting of an amino acid sequence as set forth in SEQ ID NO:1, in or on the cell, tissue or organ or portion of any of the foregoing,
thereby detecting the presence of the human CD10 protein in or on the cell, tissue, organ or portion of any of the foregoing,
wherein optionally the contacting comprises use of an immunohistochemistry (IHC) assay,
and optionally the method further comprises contacting the Ab, or Ag binding fragment thereof, or monomeric or dimeric ABP, specifically bound a CD10 polypeptide, or a polypeptide or peptide comprising an antigen or an epitope consisting of an amino acid sequence as set forth in SEQ ID NO:1, with a detectable agent to indicate or signal the specific binding of the Ab, or Ag binding fragment thereof, or monomeric or dimeric ABP, to the human CD10 protein.
and optionally the detectable agent specifically binds to the Ab, or Ag binding fragment thereof, or monomeric or dimeric ABP,
and optionally the cell, tissue, organ or a portion of any of the foregoing is or comprises: an early B cell, a pro-B cell, a pre-B lymphocyte, a mature B-lymphocyte, a follicular center cell, or a cell in a lymph node germinal center, a bone marrow stem cell, a myelopoietic cell, a T lymphocyte, a parafollicular T lymphocyte, a subpopulation of parafollicular T lymphocytes, a liver bile canalicular cell, a renal glomerular cell, a proximal tubular cell, a breast myoepithelial cell, a stromal cell around or associated with an infiltrating tumor cell, a kidney cell, an epithelial cell, a leukemia cell or a carcinoma cell,
and optionally the epithelial cell is a lung, intestine, kidney, breast or placental epithelial cell, or the leukemia cell is an acute lymphoblastic leukemia (ALL) cell, or the carcinoma cell is a tumor cell derived from an epithelial cell, or the tumor cell derived from an epithelial cell is a basal cell carcinoma (BCC) cell.

16: A method for detecting or diagnosing a cancer,
wherein the method comprises detecting expression or presence of a human CD10 protein or peptide in or on a cell, tissue or organ sample using a method of claim 15, or any of the preceding claims,
wherein the detecting of the specific binding of the Ab, or Ag binding fragment thereof, or monomeric or dimeric ABP with a CD10 polypeptide, or a polypeptide or peptide comprising an antigen or an epitope consisting of an amino acid sequence as set forth in SEQ ID NO:1, in or on the cell, tissue or organ or portion of any of the foregoing, detects or diagnoses, or assists in the detection or diagnosis of, the cancer,
wherein optionally the cancer is: leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, a myeloma, a precursor B lymphoblastic leukemia or lymphoma, a follicular lymphoma, a Burkitt's lymphoma, diffuse large B-cell lymphoma, mantle cell lymphoma, angioimmunoblastic T-cell lymphoma, precursor T lymphoblastic leukemia or lymphoma, a non-hematolymphoid sarcoma, or a carcinoma,
and optionally the carcinoma is a renal cell carcinoma or a metaplastic breast carcinoma, or the cell, tissue or organ sample is from an individual in need thereof, or the detection comprises conducting an immunohistochemistry (IHC) assay.

17: A method for treating, ameliorating or preventing a cancer comprising first detecting or diagnosing the cancer using a method of claim 16, followed by treatment of the individual in need thereof for the treatment, amelioration or prevention of the cancer,
wherein optionally the cancer is: leukemic cell cancer of pre-B phenotype, acute lymphoblastic leukemia (ALL), angioimmunoblastic T cell lymphoma, Burkitt lymphoma, chronic myelogenous leukemia in blast crisis, diffuse large B-cell lymphoma, hairy cell leukemia, a myeloma, a precursor B lymphoblastic leukemia or lymphoma, a follicular lymphoma, a Burkitt's lymphoma, diffuse large B-cell lymphoma, mantle cell lymphoma, angioimmunoblastic T-cell lymphoma, precursor T lymphoblastic leukemia or lymphoma, non-hematolymphoid sarcoma, or carcinoma,
and optionally the carcinoma is a renal cell carcinoma or a metaplastic breast carcinoma, or the carcinoma is a tumor cell derived from an epithelial cell, or the tumor cell derived from an epithelial cell is a basal cell carcinoma.

18-19. (canceled)

20: A kit comprising: a chimeric or recombinant antibody of claim 1,
wherein optionally the kit comprises components needed for an immunohistochemistry (IHC) assay, and/or comprises instructions for practicing a method of any of the preceding claims.


SEQUENCE LISTING (3 sequences)

SEQ ID NO: 1 - protein, 117 aa, artificial sequence
  synthetic peptide
EVQLQQSGPDLVKPGASVKMSCKASGYTFTDYFMKWVKQSHGKSLEWIGDINPNNGDTFYNQKFKGKATLTVDKSSSTAYMQLSSLTSEDSAVYYCAKGGFNPGDYWGQGTTLTVSS

SEQ ID NO: 2 - protein, 112 aa, artificial sequence
  synthetic polypeptide
DAVLTQAPLSLPVSLGDQASISCRSSQSLVHRNGNTYLHWFLQRPGQSPKLLIDKVSNRFSGVPDRFSGSGSGTDFTLKISRVEAEDLGVYFCSQSTHVPLTFGAGTKLELK

SEQ ID NO: 3 - protein, 750 aa, homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW